(19) United States
(12) Patent Application Publication
(10) Pub. No.: US 2026/0369357 A1
(43) Pub. Date: Nov. 19, 2026

(54) UMI COLLAPSING

(71) Applicant: Illumina, Inc., San Diego, CA (US)

(72) Inventors: Chen Zhao, San Diego, CA (US); Tingting Jiang, San Diego, CA (US); Adam Birnbaum, San Diego, CA (US); Rami Mehio, San Diego, CA (US); Severine Catreux, San Diego, CA (US); Seong Won Cha, San Diego, CA (US)

(73) Assignee: Illumina, Inc., San Diego, CA (US)

(21) Appl. No.: 19/668,727

(22) Filed: May 5, 2026

Related U.S. Application Data
(63) Continuation of application No. 17/748,455, filed on May 19, 2022, now abandoned.
(60) Provisional application No. 63/190,716, filed on May 19, 2021.

(51) Int. Cl. G16B 30/10; C12N 15/10; G16B 40/20

(52) U.S. Cl. CPC G16B 30/10; C12N 15/1065; G16B 40/20

(57) ABSTRACT

Disclosed herein include systems, devices, and methods for grouping sequence reads and collapsing families of sequence reads that originate from the same DNA molecules using UMIs. In some embodiments, the methods and systems described herein utilize a probabilistic model for UMI collapsing, in which the probability of UMI transition can be calculated and families of sequence reads can be merged based on likelihood ratio.

### RELATED APPLICATIONS

This application is a continuation of U.S. patent application Ser. No. 17/748,455, filed May 19, 2022, which claims the benefit under 35 U.S.C. § 119 (e) of U.S. Provisional Patent Application Ser. No. 63/190,716, filed May 19, 2021, the entire contents of each of which are incorporated by reference herein.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### REFERENCE TO SEQUENCE LISTING

The present application is being filed along with a Sequence Listing in electronic format. The Sequence Listing is provided as a file entitled 47CX-311974-US2_Sequence_Listing, created May 5, 2026, which is 7,368 bytes in size. The information in the electronic format of the Sequence Listing is incorporated herein by reference in its entirety.

### BACKGROUND

### Field

The present disclosure relates generally to the field of processing sequence reads, for example, grouping sequence reads.

### Description of the Related Art

To improve the error rate and accuracy of low allele frequency variant detection, different flavors of unique molecular barcode (UMI) can be added to DNA templates during library construction. Through massive deep sequencing, duplicate reads belonging to (or originating from) the same DNA templates can be grouped based on UMI sequences and consensus reads can be generated to remove error from sample processing, library preparation or sequencing. However, there can be errors in UMI sequences, or artifacts of UMI jumping can occur during library construction and sequencing. There is a need for methods that can better group duplicate reads belonging to the same DNA templates.

### SUMMARY

Disclosed herein include methods of grouping sequence reads. A method of grouping sequence reads can include grouping sequence reads into families of sequence reads; and merging (or collapsing) families of sequence reads. In some embodiments, a method for grouping sequence reads is under control of a processor (e.g., a hardware processor or a virtual processor) and comprises: receiving a plurality of sequence reads each comprising a fragment sequence and a unique molecular identifier (UMI) sequence (or an identifier sequence). The method can comprise: aligning sequence reads of the plurality of sequence reads to a reference sequence (e.g., a reference genome sequence) using the fragment sequences of the sequence reads. The method can comprise: grouping sequence reads of the plurality of sequence reads into a plurality of families of sequence reads based on the UMI sequences and positions of the fragment sequences of the sequence reads aligned to the reference sequence. The method can comprise: performing UMI statistic estimation of the plurality of families. The method can comprise: performing probability-based merging of families of the plurality of families. Performing probability-based merging can comprise: performing probability-based merging of families of the plurality of families using the results of UMI statistic estimation.

In some embodiments, performing UMI statistic estimation comprises: determining fragment (or fragment insert) size frequency, UMI jumping rate, and/or UMI frequency. Performing probability-based merging can comprises performing probability-based merging of families of the plurality of families using fragment size frequency, UMI jumping rate, and/or UMI frequency. In some embodiments, performing probability-based merging comprises: determining a relative likelihood (or probability) of the two families are derived from (or that originate from) the same original nucleic acid (e.g., DNA) molecule using the fragment size frequency, the UMI jumping rate, and/or the UMI frequency. Performing probability-based merging can comprise: determining the relative likelihood is above a merging threshold (e.g., 1). Performing probability-based merging can comprise: merging the two families of the plurality of families. In some embodiments, merging the two families comprises: merging a smaller family (e.g., with fewer sequence reads) of the two families into a larger family (e.g., with more sequence reads) of the two families.

In some embodiments, determining the relative likelihood of the two families are derived from the same original nucleic acid molecule comprises: determining a likelihood ratio of unique molecule (or family) over non-unique molecule (or family) given fragment positions. Determining the relative likelihood of the two families are derived from the same original nucleic acid molecule can comprise: determining a likelihood ratio of UMI transition for unique molecule (or family) over non-unique molecule (or family). UMI transition can be a result of UMI jumping or sequencing error. The relative likelihood is a product (e.g., multiplication product) of (i) the likelihood (or probability) ratio of unique molecule over non-unique molecule given fragment positions and (ii) the likelihood (or probability) ratio of UMI transition for unique molecule over non-unique molecule.

In some embodiments, determining the relative likelihood of the two families are derived from the same original nucleic acid molecule comprises: determining relative likelihood of the two families are derived from the same original nucleic acid molecule using a sequencing error rate (e.g., 0.001) and/or a mismatch probability (e.g., 0.25). The sequencing error rate can be predetermined. The mismatch probability can be predetermined.

In some embodiments, performing probability-based merging comprises: family identification and merging (or collapsing). Performing probability-based merging comprises can comprise: duplex identification and merging (or collapsing). In some embodiments, performing probability-based merging comprises: performing probability-based merging of families of the plurality of families using a probability map. In some embodiments, performing probability-based merging comprises: (i) for one, one or more, or each pair of families of the plurality of families, determining a relative likelihood (or probability) of the families of the pair are derived from the same original nucleic acid molecule. Performing probability-based merging can comprise: (ii) for the pair of families with the highest relative likelihood (or probability), if the relative likelihood of the families in the pair with the highest relative likelihood (or probability) are derived from the same original nucleic acid molecule is above a merging threshold (e.g., 1), then merging the families. In some embodiments, wherein performing probability-based merging further comprises: (iii) repeating (i) and (ii) until the relative likelihood of the families in the pair with the highest relative likelihood (or probability) is not above the merging threshold

In some embodiments, performing UMI statistic estimation comprises: performing UMI statistic estimation on a subset of families of the plurality of families. The subset of families can comprise at least 50,000 families of the plurality of families. The subset of families can comprise at least 10% of families of the plurality of families. The plurality of families (e.g., before probability-based merging or after probability-based merging) comprises at least 500,000 families. The plurality of families before probability-based merging is performed can comprise at least 10% more families than the plurality of families after probability-based merging is performed. In some embodiments, one, one or more, or each family of the plurality of families before (or after) merging comprises at least 1 sequence read (e.g., at least 5 sequence reads) of the plurality of sequence reads.

In some embodiments, one, one or more, or each of the plurality of sequence reads comprises a second UMI sequence. The UMI sequence can be 5′ to the fragment sequence. The second UMI sequence can be 3′ to the fragment sequence. Alternatively, the UMI sequence can be 3′ to the fragment sequence. The second UMI sequence can be 5′ to the fragment sequence.

In some embodiments, the UMI sequence is 4-20 bases in length. The second UMI sequence can be 4-20 bases in length. The UMI sequence and the second UMI sequence can have different lengths. The UMI sequence and the second UMI sequence can have an identical length. The UMI sequence and the second UMI sequence can be different. The UMI sequence and the second UMI sequence can be identical. The UMI sequences can be random. The UMI sequences can be non-random.

In some embodiments, the method comprises: subsequent to performing probability-based merging, for one, one or more, or each of the plurality of families, determining a consensus fragment sequence of the family, a position of the consensus fragment sequence aligned to the reference sequence, and/or a consensus UMI sequence of the family. The method can comprise: aligning the consensus fragment sequence to the reference sequence. In some embodiments, the method comprises: determining a fragment sequence and/or a UMI sequence of the original nucleic acid molecule from which the sequence reads of the family are derived. The method can comprise: aligning the fragment sequence to the reference sequence.

In some embodiments, the method comprises: creating a file or a report and/or generating a user interface (UI) comprising a UI element representing or comprising, for one, one or more, or each of the plurality of families, (i) the family. The file or report and/or the UI element can represents or comprise (ii) sequence reads of the family, fragment sequences of the family, and/or UMI sequences of the family. The file or report and/or the UI element can represents or comprise (iii) a consensus fragment sequence of the family, a position of the consensus fragment sequence aligned to the reference sequence, and/or a consensus UMI sequence of the family.

In some embodiments, the plurality of sequence reads comprises fragment sequences that are about 50 base pairs to about 1000 base pairs in length each. The plurality of sequence reads can comprise paired-end sequence reads and/or single-end sequence reads. The plurality of sequence reads can be generated by whole genome sequencing (WGS), e.g., clinical WGS (cWGS).

In some embodiments, the plurality of sequence reads is generated from a sample. The sample can be obtained from a subject. The sample can be generated from another sample obtained from a subject. The other sample can be obtained directly from the subject. The sample can comprise cells, cell-free DNA, cell-free fetal DNA, circular tumor DNA, amniotic fluid, a blood sample, a biopsy sample, or a combination thereof.

Disclosed herein include systems for grouping sequence reads (which can include grouping sequence reads into families of sequence reads; and merging (or collapsing) families of sequence reads). In some embodiments, a system of grouping sequence reads comprises: non-transitory memory configured to store executable instructions. The non-transitory memory can be configured to store a plurality of sequence reads each comprising a fragment sequence and a unique molecular identifier (UMI) sequence (or an identifier sequence). The system can comprise: a processor (e.g., a hardware processor or a virtual processor) in communication with the non-transitory memory. The hardware processor can be programmed by the executable instructions to perform: aligning sequence reads of the plurality of sequence reads to a reference genome sequence using the fragment sequences of the sequence reads. The hardware processor can be programmed by the executable instructions to perform: grouping sequence reads of the plurality of sequence reads into a plurality of families of sequence reads based on the UMI sequences positions of the fragment sequences of the sequence reads aligned to the reference genome sequence. The hardware processor can be programmed by the executable instructions to perform: performing probability-based merging of families of the plurality of families.

In some embodiments, performing probability-based merging comprises: performing UMI statistic estimation of the plurality of families. In some embodiments, performing UMI statistic estimation comprises: determining fragment (or fragment insert) size frequency, UMI jumping rate, and/or UMI frequency. Performing probability-based merging can comprise: performing probability-based merging of families of the plurality of families using fragment size frequency, UMI jumping rate, and/or UMI frequency.

In some embodiments, performing probability-based merging comprises: determining a relative likelihood (or probability) of the two families are derived from the same original nucleic acid molecule using the fragment size frequency, the UMI jumping rate, and/or the UMI frequency. Performing probability-based merging can comprise: determining the relative likelihood is above a merging threshold. Performing probability-based merging can comprise: merging the two families of the plurality of families. Merging the two families comprises: merging a smaller family (e.g., with fewer sequence reads) of the two families into a larger family (with more sequence reads) of the two families.

In some embodiments, the relative likelihood of the two families are derived from the same original nucleic acid molecule is a product (e.g., a multiplication product) of (i) a likelihood ratio of unique molecule (or family) over non-unique molecule (or family) given fragment positions, and (ii) a likelihood ratio of UMI transition (e.g., caused by UMI jumping or sequencing error) for unique molecule over non-unique molecule. The hardware processor can be programmed by the executable instructions to perform: determining the likelihood ratio of unique molecule over non-unique molecule given fragment positions. The hardware processor can be programmed by the executable instructions to perform: determining the likelihood ratio of UMI transition for unique molecule over non-unique molecule.

In some embodiments, determining the relative likelihood of the two families are derived from the same original nucleic acid molecule comprises: determining relative likelihood of the two families are derived from the same original nucleic acid molecule using a sequencing error rate (e.g., 0.001) and/or a mismatch probability (e.g., 0.25). The sequencing error rate can be predetermined. The mismatch probability can be predetermined.

In some embodiments, performing probability-based merging comprises: family identification and merging (or collapsing). Performing probability-based merging can comprise: duplex identification and merging (or collapsing). In some embodiments, wherein performing probability-based merging comprises: performing probability-based merging of families of the plurality of families using a probability map. In some embodiments, performing probability-based merging comprises: (i) for one, one or more, or each pair of families of the plurality of families, determining a relative likelihood (or probability) of the families of the pair are derived from the same original nucleic acid molecule. Performing probability-based merging can comprise: (ii) for the pair of families with the highest relative likelihood (or probability), if the relative likelihood (or probability) of the families in the pair with the highest relative likelihood (or probability) are derived from the same original nucleic acid molecule is above a merging threshold, then merging the families. Performing probability-based merging can further comprise: (iii) repeating (i) and (ii) until the relative likelihood (or probability) of the families in the pair with the highest relative likelihood is not above the merging threshold

In some embodiments, performing UMI statistic estimation comprises: performing UMI statistic estimation on a subset of families of the plurality of families. The subset of families can comprise at least 50,000 families of the plurality of families. The subset of families can comprise at least 10% of families of the plurality of families. The plurality of families (e.g., before probability-based merging or after probability-based merging) comprises at least 500,000 families. The plurality of families before probability-based merging is performed can comprise at least 10% more families than the plurality of families after probability-based merging is performed. In some embodiments, one, one or more, or each family of the plurality of families before (or after) merging comprises at least 1 sequence read (e.g., at least 5 sequence reads) of the plurality of sequence reads.

In some embodiments, one, one or more, or each of the plurality of sequence reads comprises a second UMI sequence. The UMI sequence can be 5′ to the fragment sequence. The second UMI sequence can be 3′ to the fragment sequence. Alternatively, the UMI sequence can be 3′ to the fragment sequence. The second UMI sequence can be 5′ to the fragment sequence.

In some embodiments, the UMI sequence is 4-20 bases in length. The second UMI sequence can be 4-20 bases in length. The UMI sequence and the second UMI sequence can have different lengths. The UMI sequence and the second UMI sequence can have an identical length. The UMI sequence and the second UMI sequence can be different. The UMI sequence and the second UMI sequence can be identical. The UMI sequences can be random. The UMI sequences can be non-random.

In some embodiments, wherein the hardware processor is programmed by the executable instructions to perform: subsequent to performing probability-based merging, for one, one or more, or each of the plurality of families, determining a fragment sequence (or a consensus fragment sequence) of the family, a position of the fragment sequence aligned to the reference genome sequence, and/or a UMI sequence of the family. The hardware processor can be programmed by the executable instructions to perform: aligning the fragment sequence of the family to the reference sequence. The hardware processor can be programmed by the executable instructions to perform: determining a fragment sequence and/or a UMI sequence of the original nucleic acid molecule from which the sequence reads of the family are derived. The method can comprise: aligning the consensus fragment sequence to the reference sequence.

In some embodiments, wherein the hardware processor is programmed by the executable instructions to perform: creating a file or a report and/or generating a user interface (UI) comprising a UI element representing or comprising, for one, one or more, or each of the plurality of families, (i) the family, (ii) sequence reads of the family, fragment sequences of the family, and/or UMI sequences of the family, and/or (iii) a fragment sequence of the family, a position of the fragment sequence aligned to the reference genome sequence, and/or a UMI sequence of the family.

In some embodiments, the plurality of sequence reads comprises fragment sequences that are about 50 base pairs to about 1000 base pairs in length each. The plurality of sequence reads can comprise paired-end sequence reads and/or single-end sequence reads. The plurality of sequence reads can be generated by whole genome sequencing (WGS), e.g., clinical WGS (cWGS).

In some embodiments, the plurality of sequence reads is generated from a sample. The sample can be obtained from a subject. The sample can be generated from another sample obtained from a subject. The other sample can be obtained directly from the subject. The sample can comprise cells, cell-free DNA, cell-free fetal DNA, circular tumor DNA, amniotic fluid, a blood sample, a biopsy sample, or a combination thereof.

Also disclosed herein include a non-transitory computer-readable medium storing executable instructions, when executed by a system (e.g., a computing system), causes the system to perform any method or one or more steps of a method disclosed herein.

Details of one or more implementations of the subject matter described in this specification are set forth in the accompanying drawings and the description below. Other features, aspects, and advantages will become apparent from the description, the drawings, and the claims. Neither this summary nor the following detailed description purports to define or limit the scope of the inventive subject matter.

### BRIEF DESCRIPTION OF THE DRAWINGS

FIG. 1A shows a schematic illustration of collapsing sequence reads. FIG. 1B depicts an exemplary illustration of the basic concept of collapsing, e.g., grouping and output (or emit) of consensus.

FIG. 2 shows non-limiting exemplary embodiments of the general process for library preparation, sequencing, and UMI collapsing.

FIG. 3 depicts data related to error correction performance from different sample types.

FIG. 4 shows an exemplary illustration of a genomic locus error.

FIG. 5 depicts an exemplary illustration of current family identification methods in which error in UMIs is assumed to be caused by sequencing and caveats.

FIG. 6 depicts a UMI jumping example with an Agilent SureSelect dataset.

FIG. 7 depicts a UMI jumping example in TSO500 fusion calling.

FIG. 8 shows an illustration of current family identification methods for read collapsing and caveats.

FIG. 9 shows an illustration of dual UMI.

FIG. 10 depicts an exemplary illustration of labeled fragments for a probabilistic framework for duplicate grouping.

FIG. 11 depicts an exemplary workflow of the disclosed probabilistic framework for duplicate grouping.

FIG. 12 depicts an exemplary model of the merging process using the disclosed probabilistic framework.

FIG. 13 depicts an exemplary illustration of candidate molecules for estimation of unique molecule by position.

FIG. 14 shows exemplary probability model stat estimation of UMI jumping in dual or single UMI embodiments.

FIG. 15 shows data related to the validation of UMI jumping estimation with a single UMI.

FIG. 16 depicts models of estimation of unique molecule by UMI for random (Top) or non-random (Bottom) UMI types. Also, See, Table 1.

FIG. 17 depicts exemplary illustration of duplex collapsing with single UMI.

FIG. 18 depicts data related to enhanced performance of error correction using the presently disclosed methods (DRAGEN v. Fulcrum genomics tools (Fgbio)).

FIG. 19 depicts data related to Pileup vs variant caller (VC) Sensitivity.

FIG. 20 shows a histogram of Truth Challenge Benchmark Data variant mutant support using DRAGEN vs Fgbio.

FIG. 21 depicts a receiver operator characteristic (ROC) curve of the impact on SNP variant calling: DRAGEN UMI+DRAGEN VC. Shown are results using the positional and probability-based models disclosed herein.

FIG. 22 depicts an ROC curve of the impact on non-SNP variant calling: DRAGEN UMI+DRAGEN VC. Shown are results using the positional and probability-based models disclosed herein.

FIG. 23 depicts an ROC curve of the impact on SNP variant calling: DRAGEN UMI+DRAGEN VC. Shown here are the results from probability models only.

FIG. 24 depicts an ROC curve of the impact on non-SNP variant calling: DRAGEN UMI+DRAGEN VC. Shown here are the results from probability models only.

FIG. 25 depicts an ROC curve of the impact on SNP variant calling: DRAGEN UMI+CG VC (LQ only). Shown are results using probability based models.

FIG. 26 depicts an ROC curve of the impact on non-SNP variant calling: DRAGEN UMI+CG VC (LQ only). Shown are results using probability based models.

FIG. 27 depicts data related to insertion/deletion (indel) error rate.

FIG. 28 depicts a flow diagram of an exemplary embodiment of the UMI calling methods disclosed herein.

FIG. 29 depicts a flow diagram of an exemplary embodiment of methods for identifying collapsible regions.

FIG. 30 depicts a flow diagram of exemplary embodiments of methods for generating consensus reads.

FIG. 31 depicts a flow diagram of exemplary embodiments for scanning for collapsible regions.

FIG. 32 shows an illustration of sequences collapsing using positional and UMI information.

FIG. 33 depicts a diagram related to UMI metrics for read pairs with duplex UMI. Also, See, Table 6.

FIG. 34 shows a diagram related to UMI error corrections. Also, See, Table 6.

FIG. 35 shows a diagram related to UMI metrics related to UMI collapsible regions. Also, See, Table 6.

FIG. 36 is a flow diagram showing an exemplary method of grouping sequence reads. Grouping sequence reads can include grouping sequence reads, based on UMI sequences in the sequence reads, into families. Grouping sequence reads can include merging families using a probabilistic model. Merging families of sequence reads is also referred to herein as read or UMI collapsing.

FIG. 37 is a block diagram of an illustrative computing system configured for grouping sequence reads.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

### OVERVIEW

To improve the error rate and accuracy of low allele frequency variant detection, different flavors of unique molecular barcode (UMI) can be added to DNA templates during library construction. Unique molecular identifiers (UMIs) are a type of molecular barcoding that provides error correction and increased accuracy during sequencing. These molecular barcodes are short sequences used to uniquely tag each molecule in a sample library. UMIs are used for a wide range of sequencing applications, many around PCR duplicates in DNA and cDNA. UMI deduplication is also useful for RNA-seq gene expression analysis and other quantitative sequencing methods. Sequencing with UMIs can reduce the rate of false-positive variant calls and increase sensitivity of variant detection. Since each nucleic acid in the starting material is tagged with a unique molecular barcode, bioinformatics software can filter out duplicate reads and PCR errors with a high level of accuracy and report unique reads, removing the identified errors before final data analysis. UMIs incorporate a unique barcode onto each molecule within a given sample library. By incorporating individual barcodes on each original DNA fragment, variant alleles present in the original sample (true variants) can be distinguished from errors introduced during library preparation, target enrichment, or sequencing.

Through massive deep sequencing, duplicate reads belonging to (or originating from) the same DNA templates can be grouped based on UMI sequences and consensus reads can be generated to remove error from sample processing, library preparation or sequencing. However, there can be errors in UMI sequences, or artifacts of UMI jumping can occur during library construction and sequencing. Relying only on UMI sequence can lead to under or over grouping reads causing errors in consensus read generation. In addition, merging duplicates from both strands of the DNA template can help to remove DNA sequence errors that occurred at the sample preparation level, however, it has been challenging to merge two different strands of the DNA template when there is a single UMI present. A method of read or UMI collapsing is described in U.S. Patent Application Publication No. 2020/0135298, entitled “SYSTEMS AND METHODS FOR GROUPING AND COLLAPSING SEQUENCING READS,” the content of which is incorporated herein by reference in its entirety.

UMI collapsing has mainly relied on the UMI sequence similarity and fragment position. Current algorithms only assume sequencing error has occurred if there is a difference in UMI sequence. However, this assumption does not hold, for example, for the artifact of UMI jumping. As described herein, this problem can be solved by first estimating the UMI jumping rate using a small portion of data and then applying this prior knowledge on the full data to evaluate how reads should be grouped using a probability framework. In the probability framework, the UMI sequence, UMI jumping rate, fragment size and coverage distribution are leveraged to assess the likelihood of merging reads with different UMI or different positions. With this technique, the problem of UMI jumping is resolved and can be applied universally on any UMI design. In addition, based on positional information, fragment size and coverage distribution, dual strand merging can be performed on data with a single UMI, greatly reducing the DNA error.

In some embodiments, reads are grouped by fragment alignment position. Within a small fuzzy window at each position (e.g., 1, 2, 3, 4, or 5), the reads are grouped first by exact UMI sequence, which forms a family. UMI jumping or hopping probability is estimated through insert size distribution and number of distinct UMI at certain positions. Within a fuzzy window, pair-wise likelihood ratio is calculated to assess if two families with different UMI sequences and genomic positions are derived from the same original molecule. Families with likelihood lower than threshold are merged. The default threshold is 1, for example.

Disclosed herein include methods of grouping sequence reads. A method of grouping sequence reads can include grouping sequence reads into families of sequence reads; and merging (or collapsing) families of sequence reads. In some embodiments, a method for grouping sequence reads is under control of a processor (e.g., a hardware processor or a virtual processor) and comprises: receiving a plurality of sequence reads each comprising a fragment sequence and a unique molecular identifier (UMI) sequence (or an identifier sequence). The method can comprise: aligning sequence reads of the plurality of sequence reads to a reference sequence (e.g., a reference genome sequence) using the fragment sequences of the sequence reads. The method can comprise: grouping sequence reads of the plurality of sequence reads into a plurality of families of sequence reads based on the UMI sequences positions of the fragment sequences of the sequence reads aligned to the reference sequence. The method can comprise: performing UMI statistic estimation of the plurality of families. The method can comprise: performing probability-based merging of families of the plurality of families. Performing probability-based merging can comprise: performing probability-based merging of families of the plurality of families using the results of UMI statistic estimation.

### Probabilistic Model of UMI Collapsing

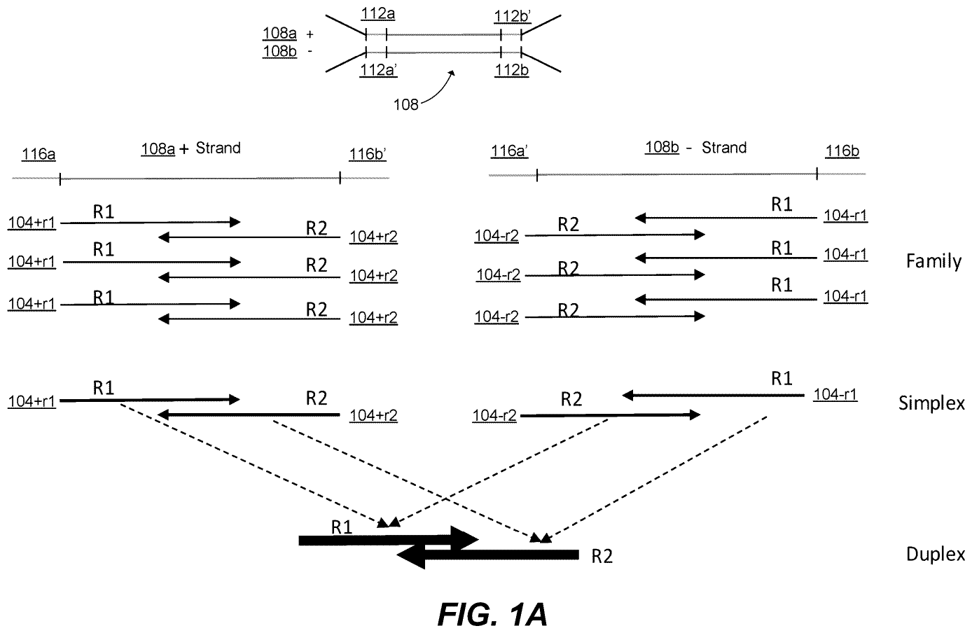

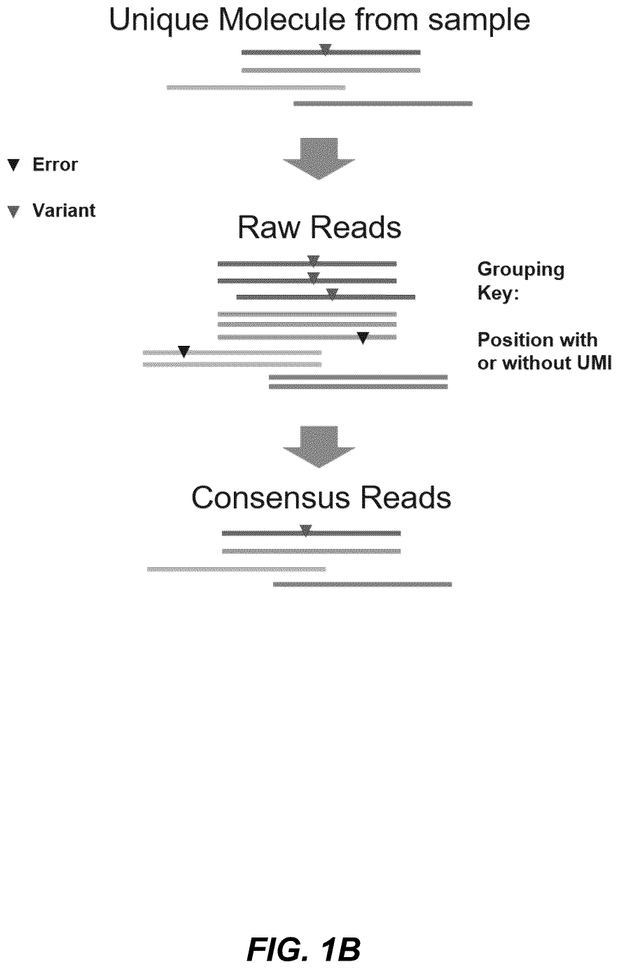

Read collapsing is a computational method that identifies nucleotide sequence reads as originating from the same source nucleic acid (e.g., DNA) molecule, and subsequently uses statistical methods to reduce spurious errors found in these sets of reads. Referring to FIG. 1A, given all the duplicate reads 104+r1, 104+r2, 104−r1, 104−r2, of the same DNA molecule 108 with a plus strand 108a and a minus strand 108b, read collapsing may include grouping those reads 104+r1, 104+r2, 104−r1, 104−r2 together. Read collapsing may include reducing spurious errors, such as with simplex collapsing to determine the nucleotide sequence of a nucleotide strand, such as the sequence of the plus strand 108a of a DNA molecule 108. Read collapsing may include inferring the sequence of the original DNA molecule 108 with high confidence, such as with duplex collapsing to determine the nucleotide sequence of a DNA molecule 108 from both the sequence of the plus strand 108a and the sequence of the minus strand 108b. The systems and methods disclosed herein may utilize a probabilistic model for grouping sequence reads (which can include merging families of sequence reads, referred to herein as read or UMI collapsing).

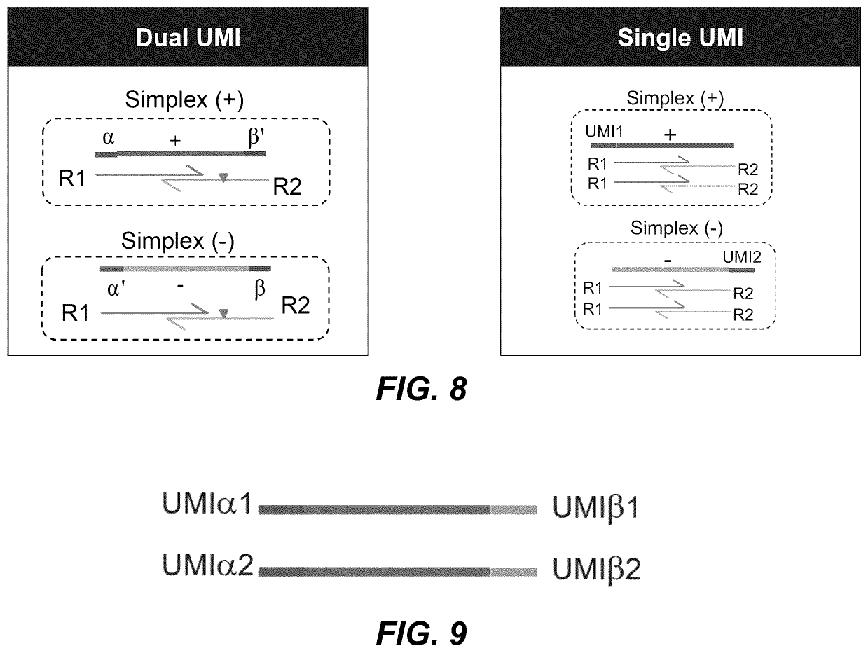

Read or UMI collapsing may produce high-quality reads. Read or UMI collapsing may require that a sample be sequenced with identifier sequences (e.g., unique identifier sequences (UMIs)) 112a, 112b′, 112a′, 112b. Such identifier sequences 112a, 112b′, 112a′, 112b can enable increased resolution when distinguishing reads and molecules that may appear very similar otherwise, though read collapsing may be performed without such identifier sequences under specific circumstances. Read collapsing may result in in-silica error reduction. Such error reduction may be useful for many applications within next generation sequencing (NGS). In some embodiments, the source nucleic acid molecules (or template) are tagged with dual UMIs as illustrated in FIG. 1A and FIG. 8, left. In some embodiments, the source nucleic acid molecules (or template) are tagged with single UMIs as illustrated in FIG. 8, right.

One application of this process is detection of variants that are only present in ultra-low allele fractions, such as in circulating tumor DNA (ctDNA). Another application is heightened variant calling specificity for clinical applications. Since read collapsing effectively combines all the duplicate observations of a DNA fragment, such as PCR duplicates of a DNA fragment, into a single representative, read collapsing has the benefit of significantly reducing the amount of data that needs to be processed downstream. Removing duplicate observations, or reads, may result in a ten-fold, or more, decrease in data size.

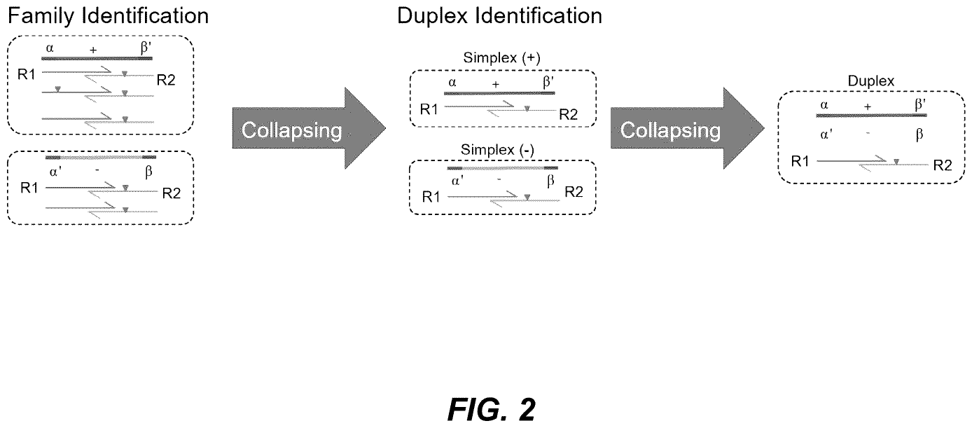

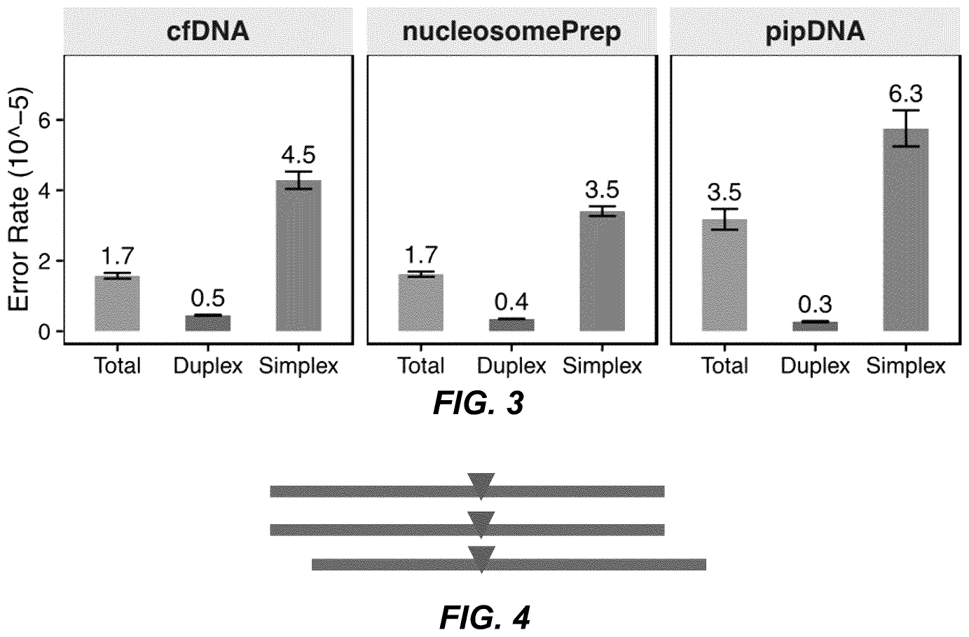

As shown in FIG. 1B, key challenges with duplicate grouping include, but are not limited to: (1) Duplicated sequence may not share the same genomic position (false negative, FN) and (2) Two unique molecules may share the same location (false positive, FP). In some embodiments, UMI helps to improve grouping accuracy. In some embodiments, the same problem of FP and FN exist with UMI. As shown in FIG. 2, read or UMI collapsing can enable error correction on single strand to remove random sequencing and PCR error, and duplex error correction can be used to remove in vitro DNA damage error (duplex collapsing). A nucleic acid or a template can be tagged with UMIs during library preparation. The resulting plus nucleic acid can have two UMIs (a on the 5′ of the nucleic acid and β′ on the 3′ of the nucleic acid). The resulting minus strand can have two UMIs (e.g., β on the 5′ of the nucleic acid and α′ on the 3′ of the nucleic acid). In some embodiments, a nucleic acid can be tagged one UMI. The tagged nucleic acid can have a fragment sequence. The tagged nucleic acid can have a UMI sequence. The tagged nucleic acid can have a second UMI sequence. UMIs Additional sequences can be added during library preparation, such as sequences for attachment to a flow cell for sequencing (e.g., P5 and P7 sequences). Read or UMI collapsing can result in error rate reduction by 10e-6-10e-5, enabling ultra-sensitive variant detection. Shown in FIG. 3 is error correction performance from simplex and duplex collapsing on circulating free DNA (cfDNA), nucleosome, and pipDNA samples. The total error rate of cfDNA was down to 10e-5, and the duplex correction yielded error rate down to 10e-6.

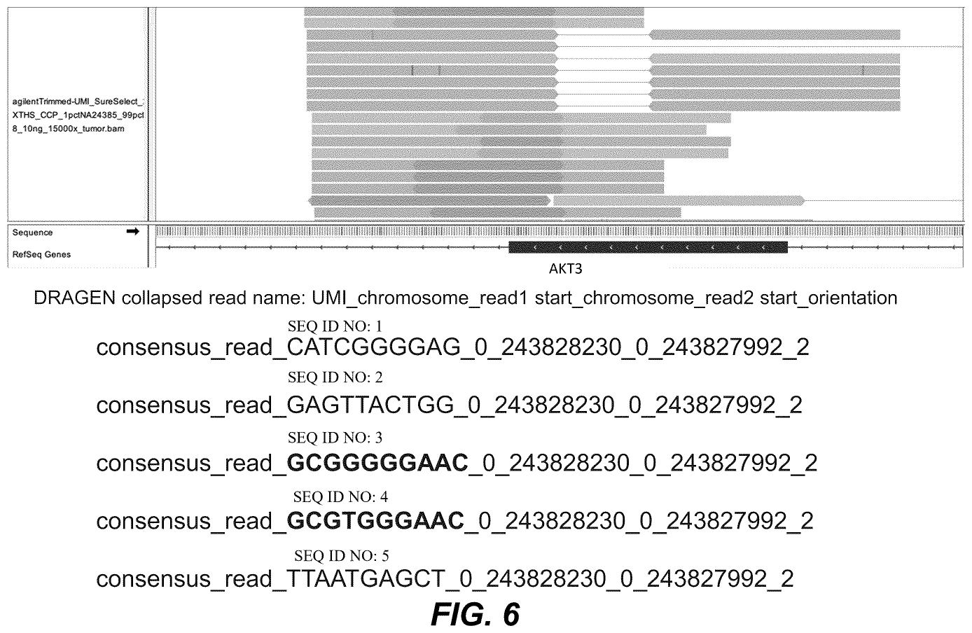

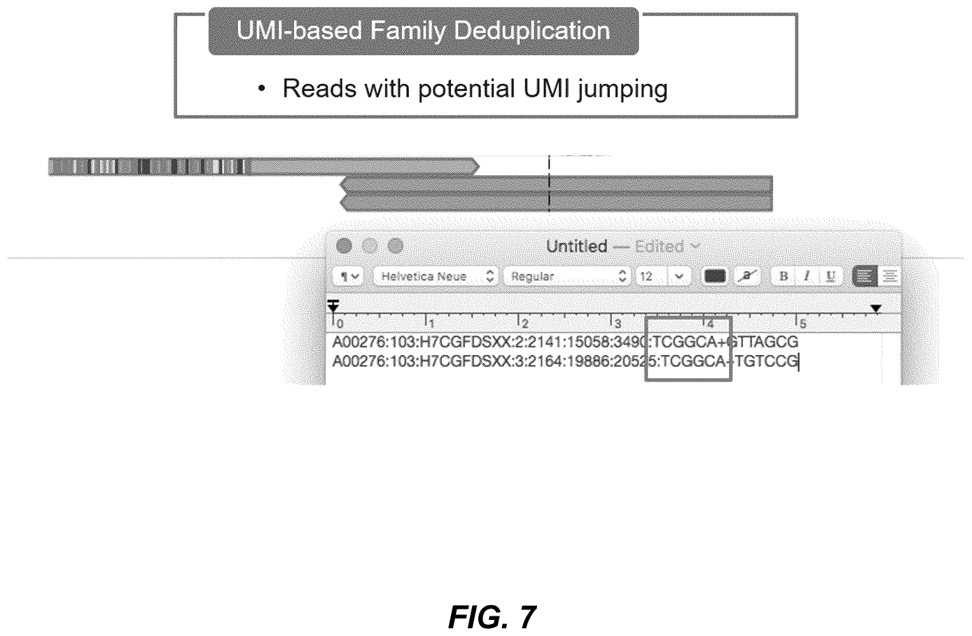

Described below are different types of UMI errors. In some embodiments, there can be a sequencing error (e.g., the UMI carries sequencing errors). For example: UMI from read1, AAT; UMI from read2, AAT; and UMI from read3, ATT. In some embodiments, there can be a genomic locus error (start/end position off by some bases), in which the UMI sequence is identical, but the position is off by a few bases (FIG. 4). In some embodiments, there is a UMI jumping error (FIG. 6-FIG. 7), in which, e.g., the UMI sequence is replaced by other sequence during PCR.

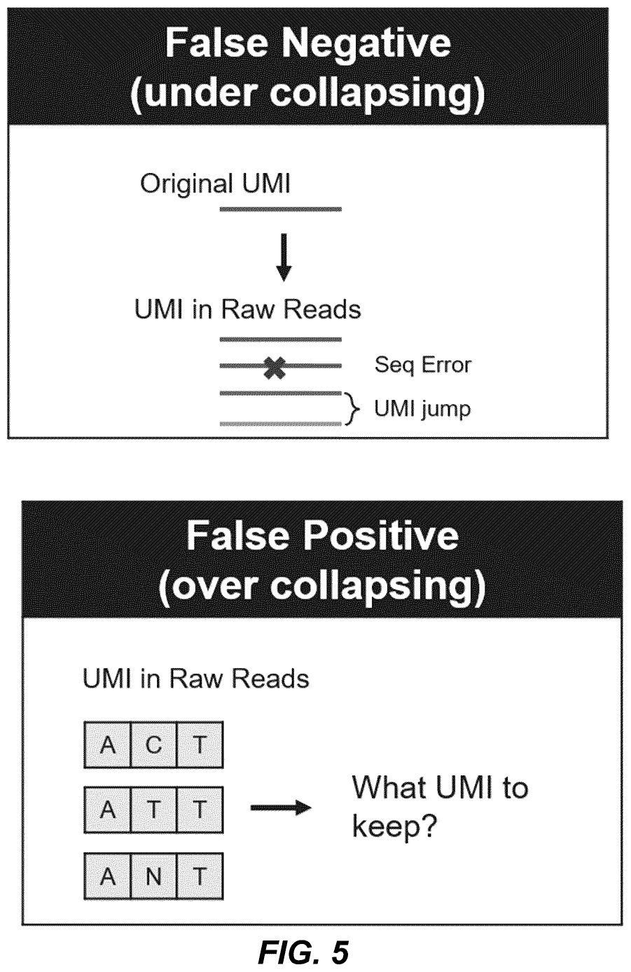

Current methods (See, e.g., FIG. 5) assume error in UMIs is caused by sequencing, and allow a mismatch of less than 1 or 2 bp. False negatives can be called due to, in some embodiments, the fact that UMI jumping rate varies from <1% to 20% in different chemistries leading to inflated error rate and incorrect collapsing. In some embodiments, false positives occur when UMI barcodes are short such as IDT/Broad design, and heuristic contextual correction may not work.

UMI correction methods can be based on or comprise heuristic rules. In some embodiments, corrected UMIs have the same start/end position and hamming distance <2 (e.g., fgbio correction). In some embodiments, the correct position is called if UMI sequence is equal and position is off by a few bases (umi-fuzzy-window-size (default=3)). Using DRAGEN option: “umi-enable-probability-model-merging=false”; Default for non-random duplex UMI.

Heuristic rules can be hard to generalize. For example, correct UMI if unique correction is nearest. If not, then, (1) Identify families where both are valid, (2) Identify families where one of UMI is invalid, only allow nearest and second-nearest, or (3) no family is identified where both are invalid, only allow nearest correction.

As shown in FIG. 7, for deduplication of potential UMI jumping read pairs, read pairs can have similar fragment alignment location (≤3 bp) and can share 1 same UMI and at least 1 same alignment. Current methods can require dual UMI for duplex collapsing (FIG. 8). A missing single end UMI can disable grouping of duplex sequence. FIG. 9 shows an illustration of dual UMI.

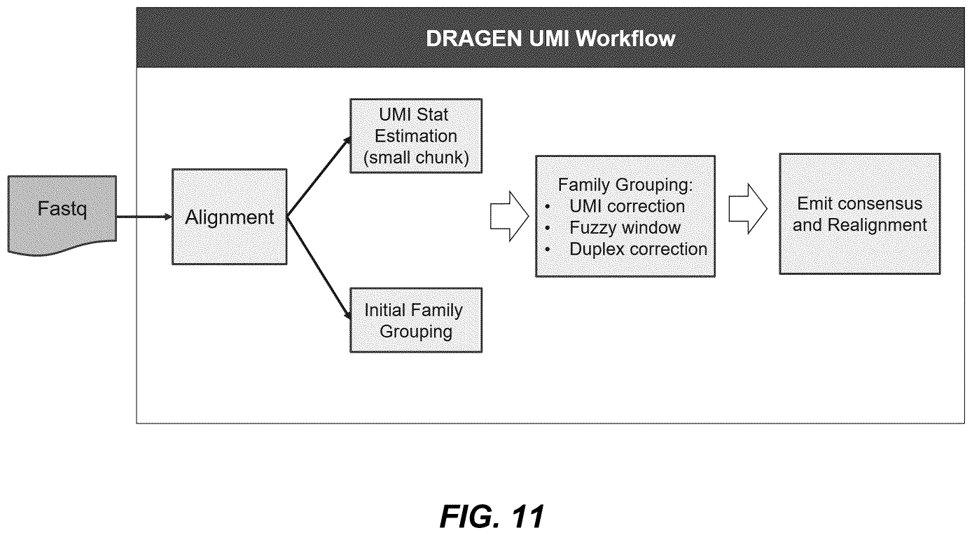

With the probabilistic models disclosed herein (FIG. 11), the probability of UMI transition can be calculated and correct UMI and merge into families can be based on likelihood ratio. In, e.g., DRAGEN pipeline option: umi-enable-probability-model-merging=true; Default for random simplex/duplex UMI.

Equations 1 and 2 below describe a probabilistic framework for duplicate grouping.

$$L = \frac{P ( C 1 = C 2 )}{P ( C 1 != C 2 )} \qquad ( 1 )$$

$$L = \frac{P ( C 1 = C 2 \vert pos ) * P ( C 1 = C 2 \vert umi )}{P ( C 1 != C 2 \vert pos ) * P ( C 1 != C 2 \vert umi )} = {L}_{pos} * {L}_{umi} \qquad ( 2 )$$

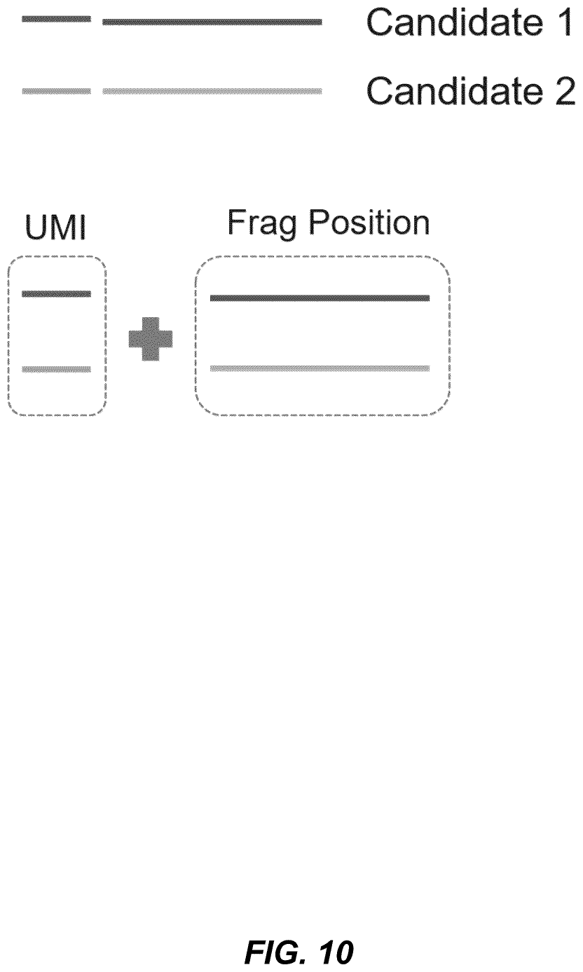

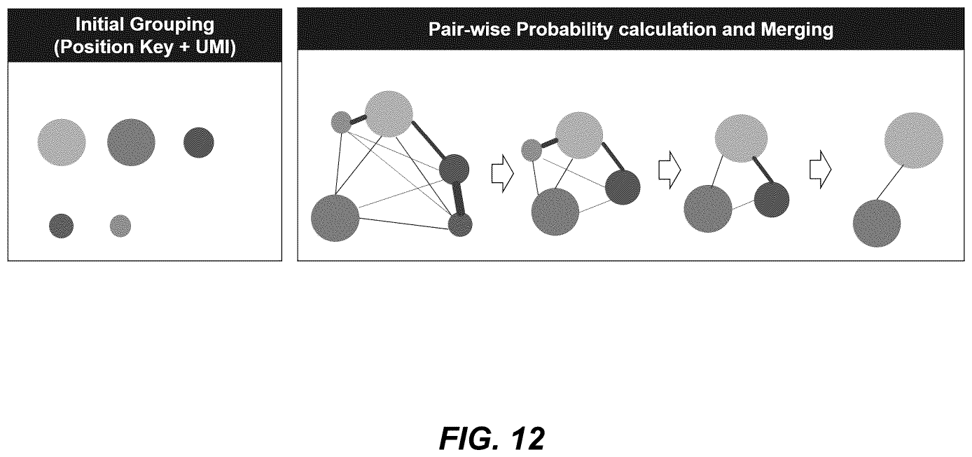

Lₚₒₛ: Likelihood ratio of unique molecule over non-unique molecule given fragment positions. Lᵤₘᵢ: Likelihood ratio of umi transition for unique molecule over non-unique molecule. Assumptions include that the UMI transition is caused by jumping or sequencing error and only larger family can jump into smaller family (C1 is larger than C2, FIG. 10). As shown in FIG. 12, initial grouping can comprise grouping reads by UMI plus position key and ordering by family size and UMI sequence. For pair-wise probability calculation and merging, pair-wise probability is computed. Only larger family can jump into smaller family, and said pair is prioritized. The pair with largest Probability (likelihood) is identified and compared with threshold. If merge is successful, probability map is recomputed until largest pair<threshold.

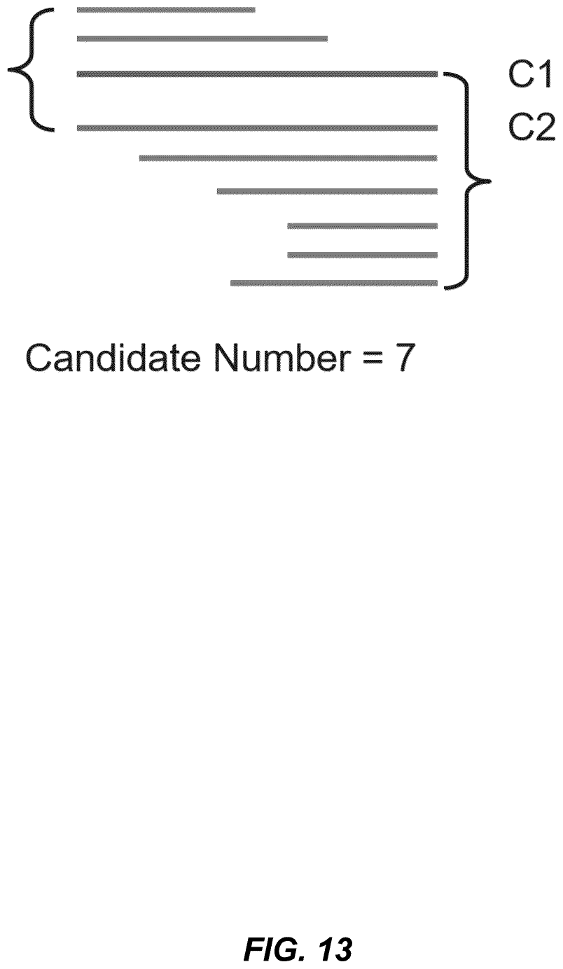

For estimation of unique molecule by position (FIG. 13), all reads can be aggregated in a region with the same start or same end as C1 and C2. Next, the frequency of insert size for C1 and C2 can be extracted. Lₚₒₛ can be estimated by binomial distribution as shown below:

$$Lpos = \frac{P ( C 1 = C 2 \vert pos )}{P ( C 1 != C 2 \vert pos )} = \frac{( \begin{matrix}7 \\ 1\end{matrix} ) \times {0.01}^{1} \times {0.99}^{6}}{( \begin{matrix}7 \\ 2\end{matrix} ) \times {0.01}^{2} \times {0.99}^{5}} = 39.9 \qquad ( 3 )$$

Where: Insert_size freq=1%, candidate number=7.

The probabilities methods disclosed herein can advantageously leverage all reads in a region rather than reads with the same start and end. In some embodiments, if C1 and C2 have shifted position, “Lₚₒₛ=Lₚₒₛ*indel error rate”. The indel error rate can be, for example, 0.001, 0.0001, or 0.00001.

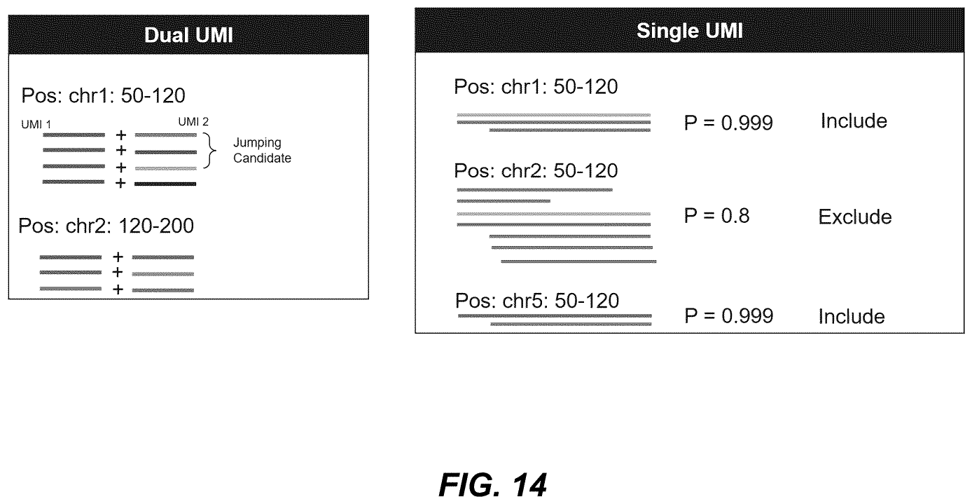

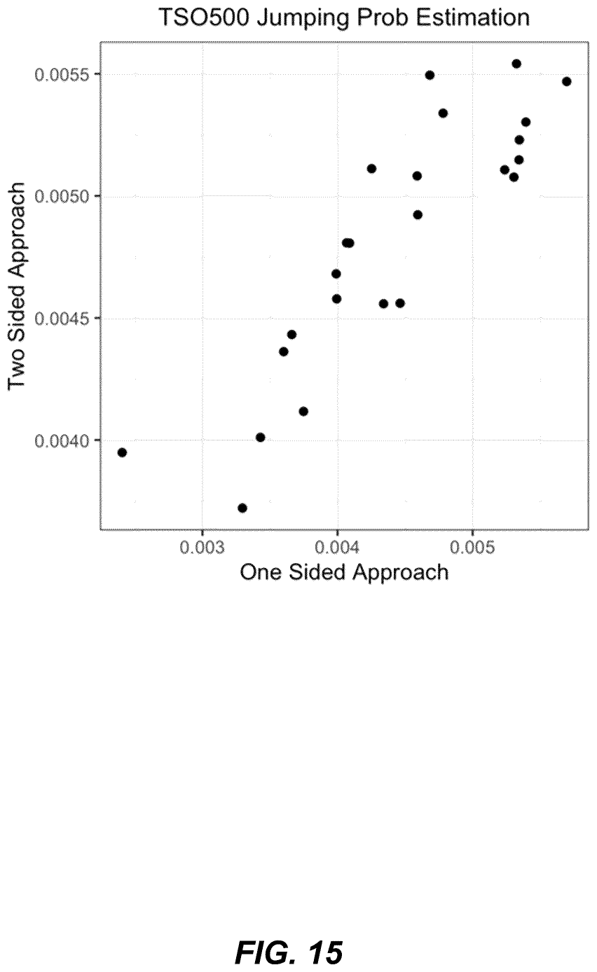

Shown in FIG. 14 are exemplary embodiments of probabilistic methods for estimating UMI jumping. For Dual UMI, any pair with one side UMI same can be considered a jumping candidate, “P(jump)=non-unique family/total family” (e.g., P(jump)=2/7). For single UMI, the probability of unique sequence per region can be estimated, P(jump) can be calculated at a region with high P(unique), “P(jump)=non-unique family/total family” (e.g., P(jumping)=1/5). FIG. 15 shows data related to the validation of UMI jumping estimation with a single UMI.

Table 1 shows exemplary formulae for calculating P(C1=C2) and P(C1!=C2), which can be in turn used to calculate L (see equation 1).

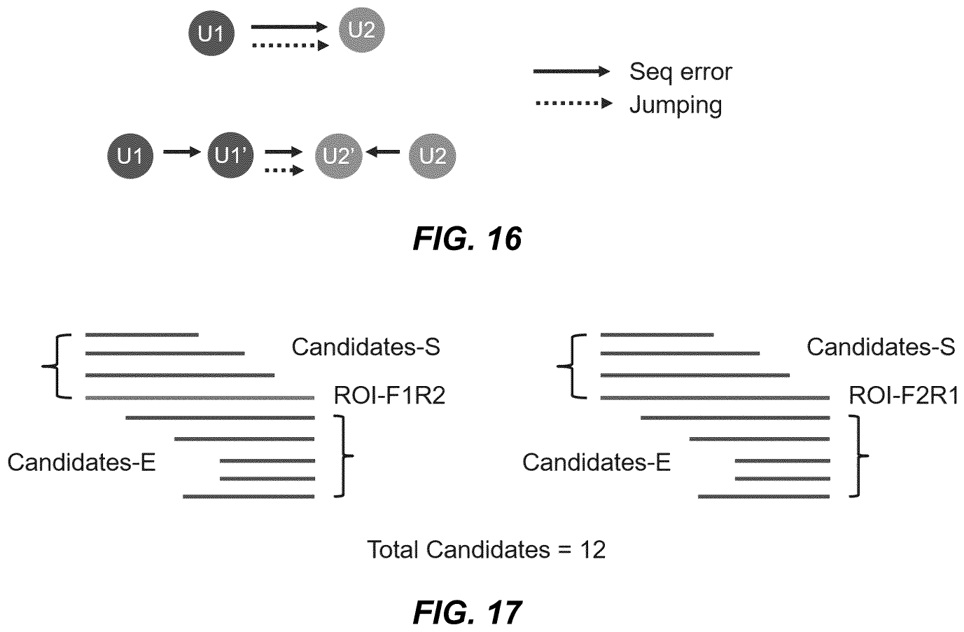

TABLE 1
ESTIMATION OF UNIQUE MOLECULE BY UMI

| | UMI | | | |
|---|---|---|---|---|
| UMI Type | Match | P(C1 = C2) | P(C1! = C2) | Model |
| Random | N | P(seq er}^{Dis} + P(jump) | P(dis) | See, |
| | | * P(dis) | | FIG. 16 |
| | Y | 1 | P(0) | Top |
| Non- | N | P(seq er}^{Dis} + P(jump) | 1-P(C1) | See, |
| random | | * P(C2) | | FIG. 16 |
| | Y | 1 | P(C1) | Bottom |

Assumption: UMI transition is caused by jumping or sequencing error. Only larger family can jump into smaller family (C1 is larger than C2).

### Equations for Lᵤₘᵢ

### Random (UMI not Matched)

$$P ( C 1 = C 2 ) = {P ( seq er )}^{Dis} + P ( jump ) * P ( dis ) = {e}^{d} + {p}_{j} \times ( \begin{matrix}l \\ d\end{matrix} ) \cdot {( 1 - {p}_{m} )}^{d} \cdot {p}_{m}^{1 - d} \qquad ( 4 )$$

Where, e=sequencing error rate, d=hamming distance, l=UMI length without N base, pⱼ=UMI jumping probability (from stat estimate), pₘ=mismatch probability=0.25.

$$P ( C 1 \neq C 2 ) = P ( dis ) = ( \begin{matrix}l \\ d\end{matrix} ) \cdot {( 1 - {p}_{m} )}^{d} \cdot {p}_{m}^{l - d} \qquad ( 5 )$$

### Non-Random (UMI not Matched)

P(C1=C2)=P(seq er)^{Dis}+P(jump)*P(C)=e^{d}+pⱼ×p_{c} (6)

Where p_{c}=frequency of UMI C (from stat estimate).

P(C1≠C2)=1−P(C)=1−p_{c} (7)

### Equations for Lₚₒₛ

$${L}_{pos} = \frac{P ( A = B )}{P ( A \neq B )} \qquad ( 8 )$$

$$P ( A = B ) = Binom ( 1 ; {N}_{f} , {f}_{i} ) \times {e}_{i}^{d} \qquad ( 9 )$$

$$Where , d = fragment positional difference = \{ \begin{matrix}2 , & if start and end position different \\ 1 , & else if start or end position different \\ 0 , & otherwise\end{matrix}$$

$${e}_{i} = indel error rate , {N}_{f} = Numer of fragment at given position ,$$

$${f}_{i} = frequency of insertion size ( from stat estimate ) .$$

$$P ( A \neq B ) = 1 - {\sum }_{k = 0}^{1} Binom ( k ; {N}_{f} , {f}_{i} ) \qquad ( 10 )$$

$$Binom ( k ; n , p ) = \frac{n !}{k ! ( n - k ) !} {{p}^{k} ( 1 - p )}^{n - k} \qquad ( 11 )$$

Referring to FIG. 17, after simplex collapsing, total candidates with the same start or same end are set as n. Similarly Lₚₒₛ can be estimated. In some embodiments, the sum is taken as the estimate. In some embodiments, duplex rate can be estimated first.

TABLE 2
USE CASE FOR PROBABILISTIC FRAMEWORK

| UMI Type | Use Case | Supported | Note |
|---|---|---|---|
| Non Random | NFE single UMI | Y | Disable duplex mode |
| Non Duplex | (Rhodium Program) | | as there is no duplex |
| One sided UMI | | | reads |
| Non Random | Trusight UMI | Y | |
| Duplex | Rhodium Y forked | | |
| Two sided UMI | UMI | | |
| | IDT Prism | | |
| Random | Gritstone | Y | |
| Duplex | Broad | | |
| Two sided UMI | IDT Duplex | | |
| Random | Agilent | Y | Can infer duplex based |
| Non duplex | | | on positional info |
| One sided UMI | | | |

The probability-based UMI collapsing method disclosed herein can be accurate under different UMI settings: random single UMI, nonrandom single UMI, random dual UMI, and/or nonrandom dual UMI. In some embodiments, parameters for the probability model can be fine-tuned. For example, additional statistics, such as shift probability and mismatch rate, can be made and used. Optimal threshold for the merging probability can be determined can used. In some embodiments, consensus sequencing generation can be improved. Error rate can be estimated from raw read and applied as prior for consensus read generation (e.g., estimate error rate from homopolymer region to improve indel error rate; e.g., estimate error rate from simplex read to improve duplex collapsing).

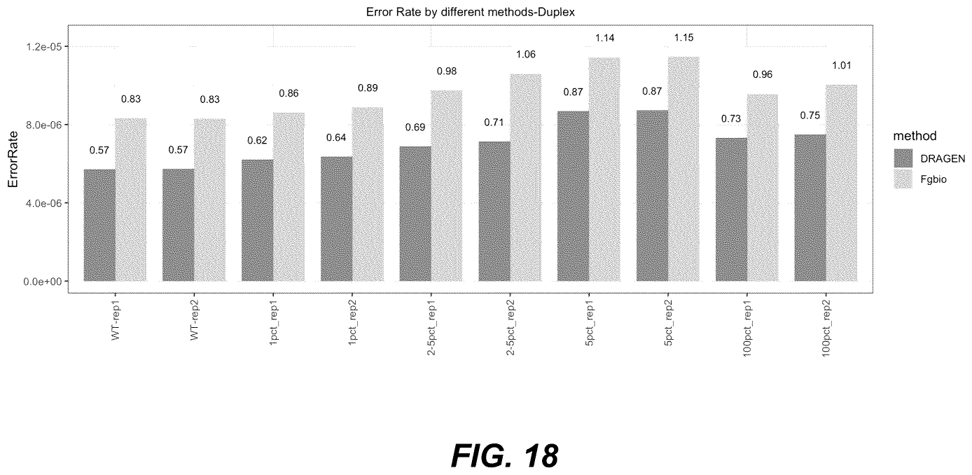

The results described below are from data using IDT duplex UMI. As shown in FIG. 18, the error correction performance on duplex reads is improved using the presently disclosed methods in DRAGEN as compared to, e.g., fulcrum (fgbio).

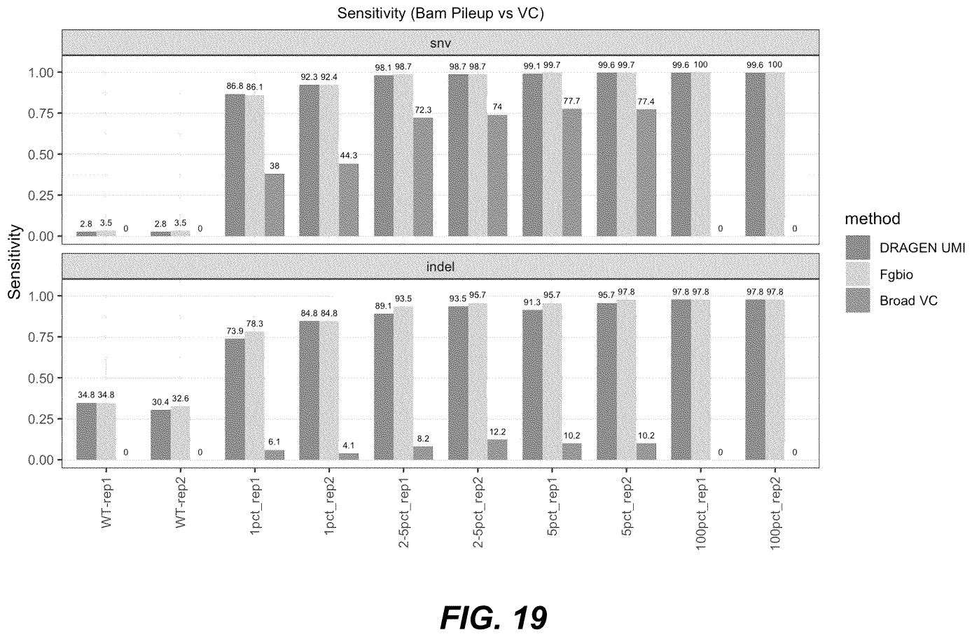

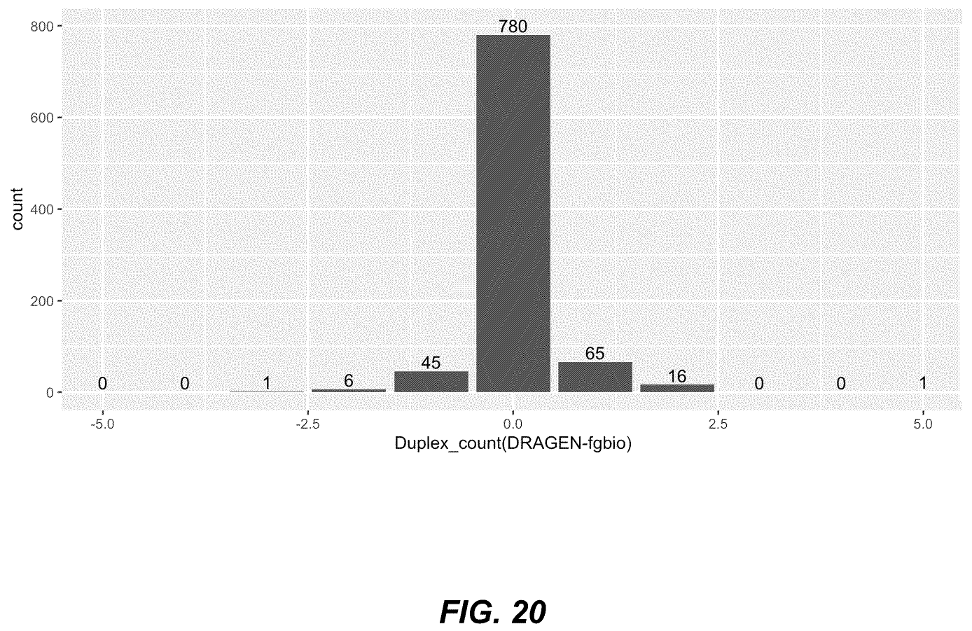

Sensitivity of DRAGEN UMI is similar to, e.g., Fgbio-duplex as shown in FIG. 19. Truth variants each with at least one duplex support are shown in FIG. 19. Missed cases may be due to alignment difference and end-masking. Low sensitivity in indel due to noisy loci in normal sample. DRAGEN calls more real support for variants than Fgbio as shown in FIG. 20. Without being bound by any particular theory, remaining cases of missed support is due to consensus generation not read grouping.

TABLE 3
DRAGEN RUN TIME (MIN:SEC)

| | DRAGEN UMI | Fgbio | |
|---|---|---|---|
| | (collapsing + | (collapsing + | DRAGEN |
| Sample ID | realignment) | realignment) | End-to-End |
| 1pct_rep1 | 13:55 | 482:34 | 22:02 |
| 100pct_HD78_rep1 | 22:04 | | 31:02 |
| 1pct_rep2 | 20:15 | | 28:30 |
| 100pct_HD78_rep2 | 25:36 | | 34:01 |
| 2-5pct_rep1 | 24:13 | | 33:03 |
| 2-5pct_rep2 | 26:43 | | 36:39 |
| 5pct_rep1 | 22:22 | | 34:03 |
| 5pct_rep2 | 25:50 | 482:98 | 34:48 |
| 100pct_HD84_rep1 | 10:03 | 369:48 | 17:05 |
| 100pct_HD84_rep1 | 18:51 | | 28:23 |

As shown above in Table 3, there is a 10-25 min per sample runtime for DRAGEN UMI. This is about 15-20 times faster than Fgbio workflow.

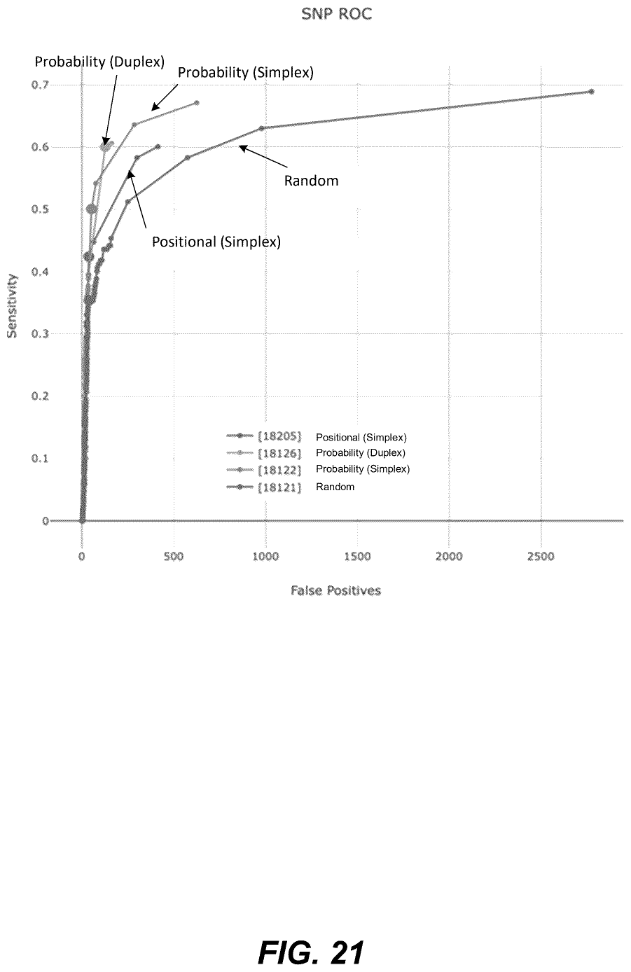

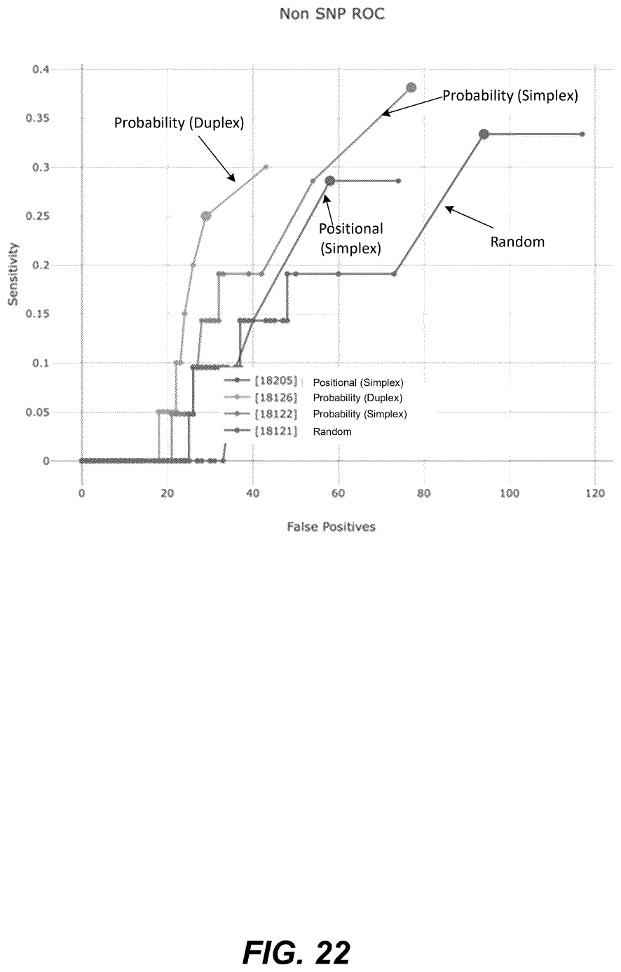

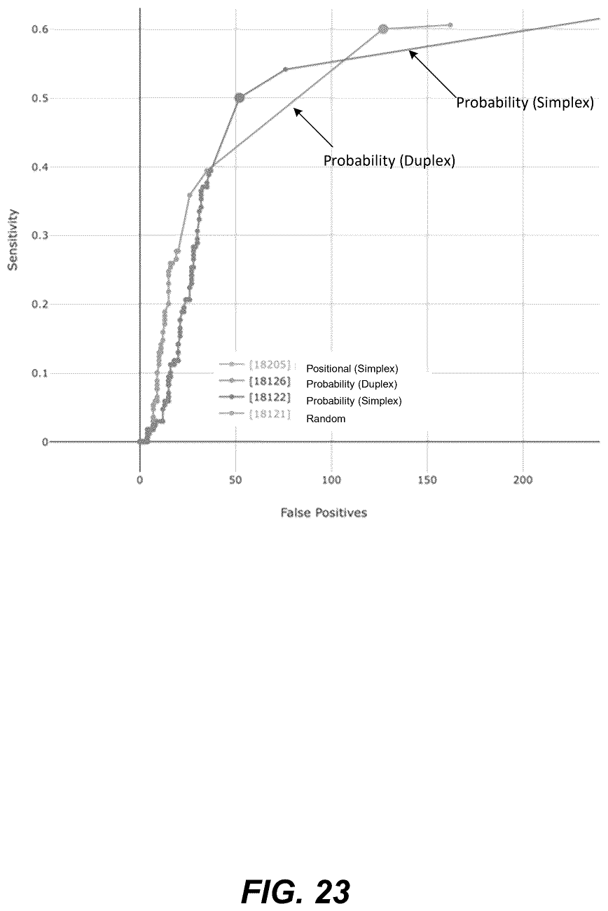

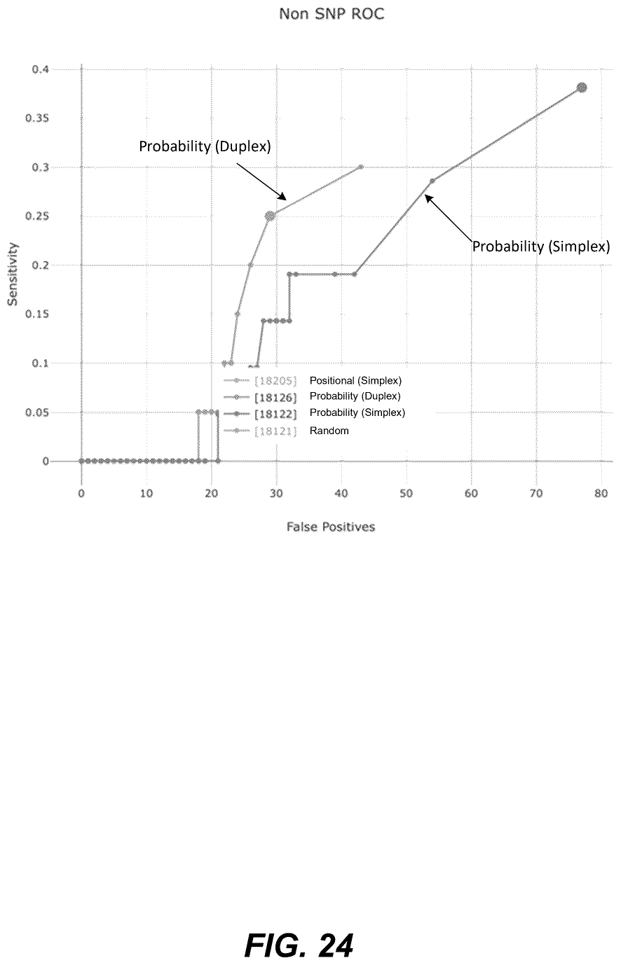

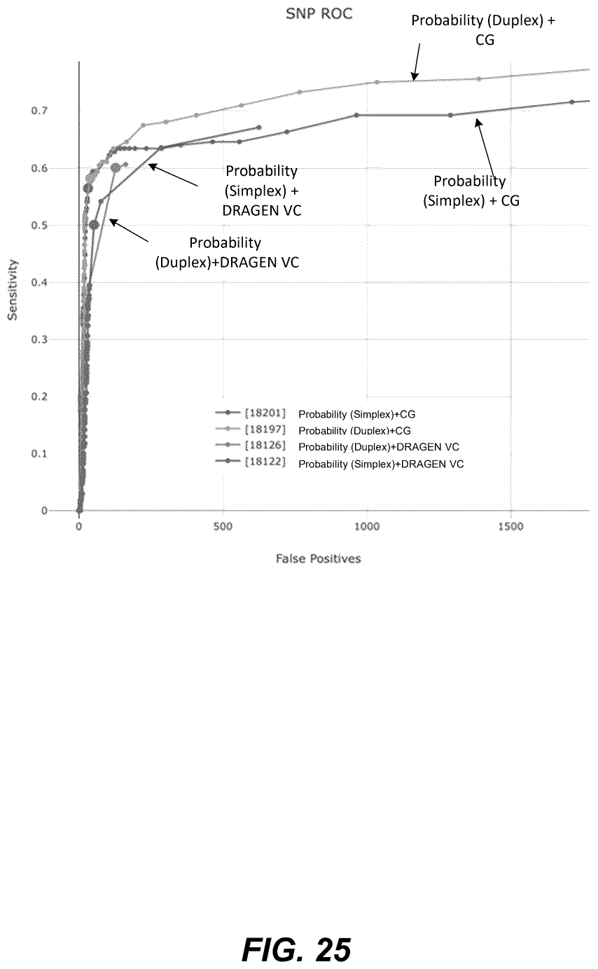

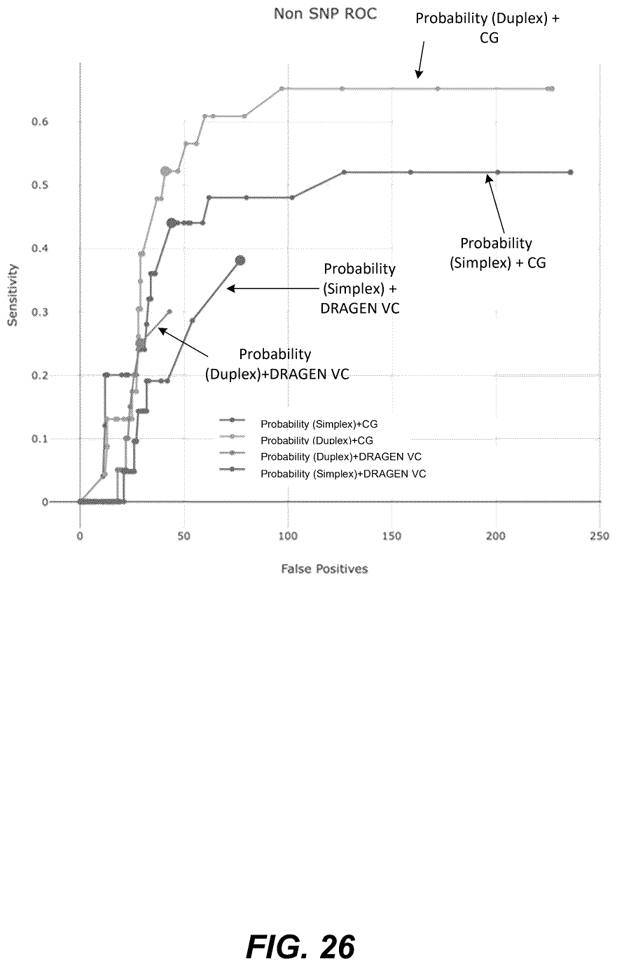

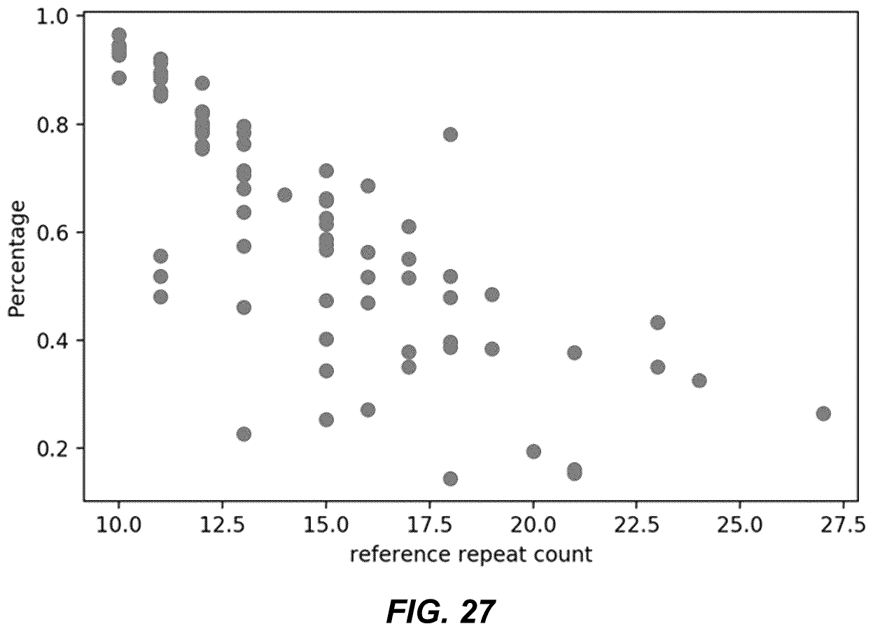

FIGS. 21-26 show the results of UMI collapsing with a probability model of the present disclosure from data using Agilent random single UMI. In some embodiments, current majority voting yields up to 80% incorrect genotypes in long repeat units (FIG. 27). In some embodiments, transition probability between different genotypes can be estimated and applied during consensus generation.

TABLE 4
PERFORMANCE OF ERROR CORRECTION

| | Median Target | Error Rate | |
|---|---|---|---|
| Collapser | Coverage | (e−5) | Note |
| Fulcrum | 698 | 10.60 | MSR = 2 |
| Agilent | 534 | 6.57 | Agilent |
| DRAGEN DeDup | 526 | 19.73 | MSR = 1 |
| DRAGEN Random | 697 | 9.01 | MSR = 2 |
| DRAGEN Probability | 606 | 8.89 | MSR = 2 |
| DRAGEN Positional | 606 | 8.91 | MSR = 2 |
| DRAGEN Probability | 437 | 6.49 | Duplex |
| Duplex | | | MSR = 2 |
| DRAGEN Positional | 437 | 6.48 | Duplex |
| Duplex | | | MSR = 2 |

### Detailed Software Design

### 1. General Design

Library preparation methods can provide the ability to attach unique identifiers (UMIs) to molecules before PCR and sequencing. This makes it possible to take post-sequenced reads, group them by UMI, and thus aggregate the evidence for what the pre-PCR fragment was. Described herein is the design of software pipeline (e.g., on DRAGEN platform from Illumina, Inc.) logic that accomplishes these tasks.

In some embodiments, the general design for DRAGEN's UMI processing is as follows: (1) Group alignments by their original source fragment, (2) Generate a single consensus read (or pair) for each source fragment, and (3) Align the consensus read and feed it into the downstream analysis pipeline (e.g., sort, variant callers).

In some embodiments, processing a full input sample through a single hashtable can run slowly. Therefore, a method was developed to identify genomic regions that may be processed independently of the other regions, and are processed in parallel.

### 2. Software Unit Design

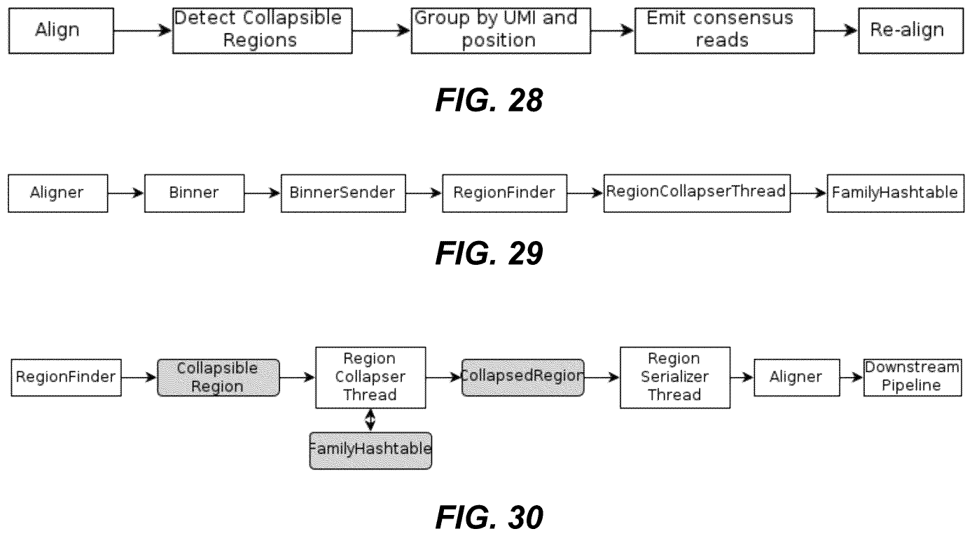

FIG. 28 shows units of the software described in this section.

### 2.1. Design Constraints

In some embodiments, the design is based on the following constraints: if the inputs are FASTQ files, the UMI tags must be contained in the read name field or provided in a separate FASTQ files; if the inputs are BAM files, the UMI tags must be contained in the read name field or in the UMI bam tag; and input FASTQ/BAM are from a paired-end run.

In some embodiments, the software can only support the following conditions: single UMIs that are less than or equal to 15 base pairs and dual UMIs that are less than or equal to 8+8 base pairs.

### 2.2. Grouping by Source Fragment: Family Hashtable

Through the PCR and sequencing process, a single original DNA fragment can, in some embodiments, lead to multiple input reads, differing from each other by sequencing errors. Described herein are methods to gather reads into groups where all of the members of the group have matching UMI, and sequences for all reads are close to identical. In some embodiments, the method for detecting sequence similarity is to use the aligner; any reads that align to the same genomic location must have a similar sequence. Thus reads can be grouped by a key comprising UMI and (clip-adjusted) pair alignment coordinates. For grouping, a hashtable of reads can be built using this key.

In some embodiments, the first stage of UMI processing is to do a normal aligner run, and to partition and sort by clip-adjusted mate coordinates. This uses a typical sort-partitioning data structure, the Binner. At the conclusion of the first alignment run, all reads have been partitioned in this Binner data structure, and then later partitions of reads can be loaded, sorted by coordinate, and independent regions for parallel processing can be identified.

### 2.3. Identifying Collapsible Regions

FIG. 29 depicts a flow diagram of an exemplary embodiment of methods for identifying collapsible regions. A group of related reads, also known as a family, can be identified as having very close alignment positions (within a “fuzzy window” of a few base-pairs), and very similar UMIs. And as coverage varies across the genome, there are many positions where it can be safely concluded that no families may be merged across that position, e.g., there are natural “break points” where family assembly can be processed independently.

In some embodiments, during this second stage of UMI processing, sort partitions can be read back into memory, sorted, and scanned for “collapsible regions”. Each “CollapsibleRegion” is assigned to a separate “RegionCollapserThread” to generate an independent set of consensus reads, in a “CollapsedRegion” data structure. The “CollapsedRegions” are put back into their intended order by a “RegionSerializerThread”, which pumps the consensus reads directly back into the DRAGEN aligner.

### 2.4. Generating Consensus Reads: Read Collapser

FIG. 30 depicts a flow diagram of exemplary embodiments of methods for generating consensus reads. As described above, the workunit for this phase of the UMI processing is the “CollapsibleRegion”. It is the job of the “RegionCollapserThread” to receive a “CollapsibleRegion”, feed all of that region's reads into a “FamilyHashtable”, and use that hashtable to generate a set of consensus reads. Details of these read-collapsing methods, including UMI matching/correction, are described below.

### 2.5. Realignment and Downstream Pipelines

The “RegionCollapserThreads” feed outputs-“CollapsedRegions”-into a single queue, where they are put into the correct output order by a single “RegionSerializerThread”. This object takes the generated reads and sends them directly into the DRAGEN aligner, and from there into any of the configured downstream systems. In some embodiments, this will typically include a conventional sort by, e.g., non-clip-adjusted alignment position, and one or more variant callers. Those downstream pipeline elements run in their normal way, without any special knowledge that the reads were generated by a UMI collapsing system.

### 2.6. Read Collapsing Algorithms

Described above is the general layout of software units that accomplish the following tasks: (1) Identify genomic regions that may be independently collapsed, (2) Group alignments by UMI and pair-alignment-position, and (3) Generate consensus reads (e.g., “collapse reads”).

The algorithms that accomplish these tasks are described in more detail below.

### 2.6.1. Collapsible Regions

Based on extensive analysis of large (TSO500) datasets, it was found that even for high depth data it is possible to find a huge number of genomic locations where one can be sure that no group of reads with matching UMIs are nearby. Thus the reads can be split into workunits for independent parallel processing, constructing separate data structures and generating consensus reads independently for each region. The algorithm by which these “CollapsibleRegions” can be identified is described below.

The goal in family construction is to group reads by clip-adjusted position and UMI. Although this will ultimately be accomplished using a hashtable-based approach (see below, the section on “Family construction”), the scan for “CollapsibleRegions” requires a sort by leftmost, clip-adjusted position. This sort is done directly downstream of the aligner, and the “RegionFinder” scans these sorted pairs.

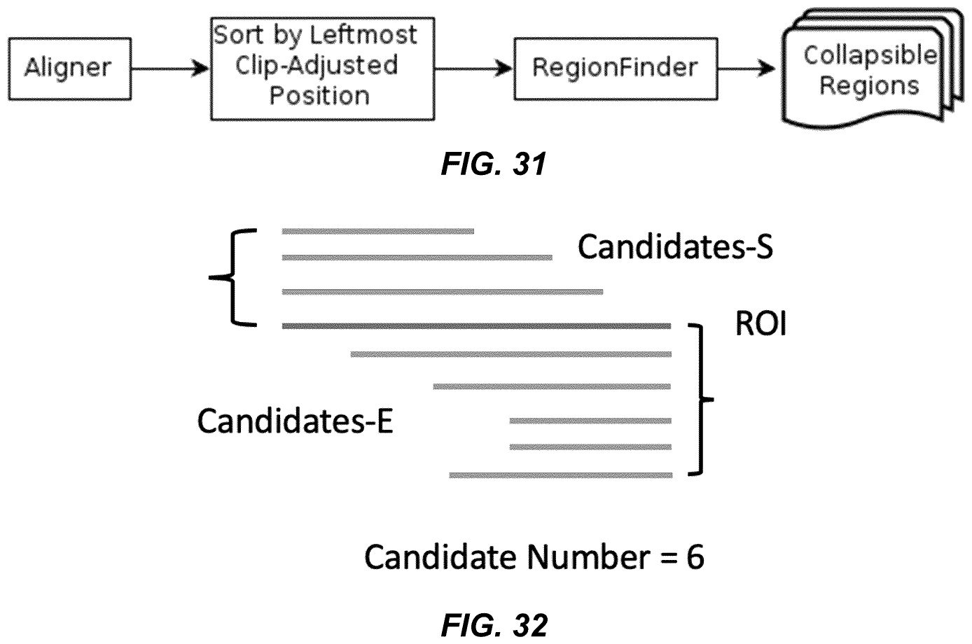

FIG. 31 depicts a flow diagram of exemplary embodiments for scanning for collapsible regions. As it scans through the sorted records, the “RegionFinder” tracks the last N (fuzzy_window+1) pair positions covered by at least some reads. As a new pair is scanned, it is checked if it can be shift-merged with any of the recent families (same left-most position and right most position difference≤fuzzy window). If so, this new position is considered to be a match, and a note is made not to split at that position.

As the scan continues, a new collapsible region will be emitted when it is found that one of the following 3 conditions are met: (1) The scan has reached a new chromosome; (2) The number of reads scanned>“minReadsPerRegion” (default 4096), and the current position does not have an ensuing match within the fuzzy window; and (3) A maximum region size is reached, e.g., number of reads scanned>“MAX_NUM_READS_WARNING” (default 500,000).

When a “CollapsibleRegion” is emitted, all accumulated reads are sent to downstream threads for family construction and corrections. Then the same scanning process is continued starting on the next available position with reads in it.

### 2.6.2. Family Construction

A Family is a grouping of sequencing data from reads that ostensibly originate from copies of the same source molecule. A Family is defined by the following information: (1) UMI, single or dual UMI noted by “+”; (2) Clip-adjusted pair coordinates, the alignment position of each mate is taken and adjusted outward beyond the 5′ end by the total amount of CIGAR soft clips; and (3) Orientation, each Family's orientation is set based on the strand direction of read1 and read2, in that order. For example, if read1 is mapped to the forward strand and read2 is mapped to the reverse strand, the orientation of the family is Forward-Reverse. During the initial scan of a “CollapsibleRegion”, reads are grouped into Families based on an exact match of these criteria.

### 2.6.3. Family Merging (UMI Correction)

After the initial construction of families, a series of criteria are considered by which families can be combined. If two families are nearby and have very similar UMIs, then it is likely that they are derived from the same input fragment. In some embodiments, there are two separate implementations of family merging: a heuristic-based implementation derived from Read Collapser (ReCo) tool from Illumina, Inc., and the presently disclosed probability-model implementation. Both implementations can apply the following three types of family merging: (1) UMI correction, in which two families with exactly the same position are combined, but are tolerably close in UMI sequence; (2) Shift-merge, in which two families with small (<fuzzy window) difference in clip-adjusted pair coordinates are merged; and (3) Duplex-merging, in which two families with complimentary orientations and matching coordinates and UMIs are combined, because they can originate from two strands of the template molecule.

### 2.7. Heuristic Based Model

In this section, the family merging procedures for the heuristic-based implementation of family merging are described, based on the ReCo tool from Illumina, Inc. Each of the described correction types may be independently enabled/disabled with commandline options.

### 2.7.1 Non-Random Dual UMI

### 2.7.1.1 UMI Correction

The UMI correction merges families with the same start-end but mismatch in UMI sequence. If the UMI code has a unique correction defined by the correction table to be the ‘true’ UMI, the corrected UMI will be assigned. For remaining families that do not have uniquely corrected UMIs, the process can work as described below.

First, families are grouped by start and end positions. Next, for each group, families with UMI1 and UMI2 combinations where both sequences are true codes are identified, and these are used as targets for correction. For each family where UMI1 and UMI2 are not both true codes, ReCo shall loop through the target families and merge the candidate family to the target if the orientations match and the any of the following apply (This is a greedy algorithm in which the first target to satisfy any of the following is taken): (1) Candidate UMI1 is the same as target UMI1, and target UMI2 is either a nearest code or second nearest code of candidate UMI2; (2) Candidate UMI2 is the same as target UMI2, and target UMI1 is either a nearest code or second nearest code of candidate UMI1 or; (3) Neither candidate UMIs match the target UMIs, however, both target UMIs are nearest codes for their respective candidate UMIs. A second nearest code is not allowed.

### 2.7.1.2. Shift-Merge

Shift correction corrects for PCR errors that result in alignment shifts. This can cause one true PCR family to informatically be viewed as multiple families with differing positions. In some embodiments, this is done according to the following steps: (1) For each family, search for other families with start and end positions within the “--umi-fuzzy-window-size” parameter, and the candidate family cannot have been shift-merged before (For example, if a family's start and end positions are {10, 20} and the window size is 3, then the following families are all likely candidates for correction: {13, 20}, {7, 23}); and (2) If two families are within a fuzzy window, determine if they can be merged. They can be merged if the orientations match and any of the following apply: (a) Both UMI1 and UMI2 match exactly. If so, merge the family with lesser total number of reads into the family with more; and (b) If one family has a good UMI combination (both UMI1 and UMI2 are true codes) and the other does not (either UMI1 or UMI2 is not a true code), determine if the bad combination can be corrected to good using the same logic in UMI Correction. If so, merge the bad family to the good.

### 2.7.1.3. Duplex Merging

UMIs are tags added to the original double stranded molecule during library preparation, and are thus propagated though the PCR family. In some embodiment, for dual UMIs, there is a separate tag for read1 and read2; the two tags (alpha and beta) in combination uniquely identify a molecule. DRAGEN UMI is able to further collapse the two consensus reads for the single strands into one consensus for the double strand via cross-family collapsing. This is possible for non-random UMIs where the UMI is in the PCR product and therefore complementary across strands.

In some embodiments, a Family (A) is the family mate of another Family (B) if all of the following are true: (1) UMI1 of Family A=UMI2 of Family B; (2) UMI2 of Family A=UMI1 of Family B; (3) Two Families, A and B, have opposite start and end positions (within the configured fuzzy window), e.g., the start position of Family A is same as, or within the fuzzy window of, the end position of Family B or the end position of Family A is same as, or within the fuzzy window of, the start position of Family B; and (4) Direction of Family A is the opposite of Family B.

### 2.7.1.4. Random Single UMI

In some embodiments, the random single UMI correction only applies UMI correction and shift-merge. The steps are as follows: (1) Loop through all the positions by order; (2) Gather family within fuzzy window and sort by family size. This is the similar procedure as finding shift-merge candidate in non-random dual UMI; and (3) Find the candidate family to merge into if the following conditions are met: (a) the candidate family has not been shift-merged before; (b) candidate target family has >“randomMergeFactor*family_size”; (c) same orientation; (d) UMI hamming distance=1 and; (e) only one target family that fits the criteria.

### 2.7.2. Positional Collapsing

In some embodiments, the UMI are not used for collapsing and reads can be collapsed based only on position.

### 2.8. Probability Model Implementation of Family Merging

In this section, the probability model-based implementation of family merging is described. There are two phases. First, statistics on UMIs in the sample are gathered to assess the likelihood that two different UMIs are derived from the same original molecular sequence. Then a second pass is made over the reads, applying the corrections.

### 2.8.1. UMI Stat Estimation

### 2.8.1.1. Fragment Insert Size Frequency

After grouping read-pairs with the exact same start-end or mismatch ≤1, UMI sequence, and strand as initial families, the frequency of insert size of the test sample can be roughly estimated. Read-pairs with low MAPQ (e.g., <60), non-properly paired or UMI with N base are excluded. Low MAPQ described herein can be, for example, <100, <75, <50, <40, <30, <20, or <10.

For the families, the user can pick the first read-pairs and define isize is the template length (standard TLEN in samtools): rightmost mapped position—leftmost mapped position. Define the lower and higher limit for insert size as LOWIZ_LIMIT (default as 50) and HIGHIZ_LIMIT (default as 500) as expected range for insert size. Counts are accumulated for different insert size in an array. If “isize LOWIZ_LIMIT”, add count to “LOWIZ_LIMIT”. If “isize>=HIGHIZ_LIMIT”, add count to “HIGHIZ_LIMIT”.

When enough counts are accumulated (in some embodiments, >50,000 families), compute the frequency of each insert size as p(insert_size).

### 2.8.1.2. UMI Jumping Rate

### 2.8.1.2.1. Two Sided UMI

With two sided UMI, a molecule with UMI jumping can produce PCR products where the UMI on one side is the same while the other side is different.
UMI1—template-UMI2
UMI1—template-UMI3

“total_family” can be set as total number of families after first round of grouping with the same start-end or mismatch ≤1, UMI sequence, and strand. Set “non_unique_family” as number of potential families due to UMI jumping. Read-pairs with low MAPQ (e.g., <60), non-properly paired, or UMI with N base are excluded. For each family, the user can pick the first read-pairs and calculate the soft-clip adjusted start-end and strand as group key.

A list can be set to accumulate families with potential UMI jumping: “family_UMI_jumping_list”. For each group key, iterate through any two families. If UMIA or UMIB are the same between two families, add both families into the list.

non_unique_family=length(family_UMI_jumping_list)−length(unique group key in family_UMI_jumping_list).

UMI jumping probability=non_unique_family/total family

### 2.8.1.2.2. One Sided UMI

With one sided UMI, the UMI jumping can look like:
UMI1—template
UMI2—template

In some embodiments, only positional information is used to determine whether a family is associated with UMI jumping. However, in some embodiments, the caveat is that it can be potentially different molecules with the same start-end which leads to overestimation of UMI jumping. To mitigate this impact, the number of families and insert size can be used to down-select the regions with only one unique molecule.

First, read-pairs with low MAPQ (e.g., <60), non-properly paired, or UMI with N base are excluded. For any family, the first read-pair is picked to compute the insert size. If “isize≤LOWIZ_LIMIT”, isize is set as “LOWIZ_LIMIT”. If “isize≥HIGHIZ_LIMIT”, set isize as “HIGHIZ_LIMIT”. Extract frequency of insert size as “p(insert_size)”. The number of candidates can be calculated as max (number of other families with same start, number of other families with same end).

In some embodiments, the number of unique molecule X is assumed to follow a binomial distribution. Binomial (n=number of candidates, p=p(insert_size)), and probability Pr(X=1) is calculated. If Pr(X=1)>threshold (e.g., 0.998), the read pair can be included, otherwise it can be excluded as the region might not, in some embodiments, contain more than 1 molecule.

### 2.8.1.3. UMI Frequency

For nonrandom UMI, after an initial round of family grouping with exact same start-end or mismatch ≤1, UMI sequence, and strand, the frequency of designed UMI sequence can be estimated. Read-pairs with low MAPQ (e.g., <60), non-properly paired, or UMI not in designed table can be discarded. For random UMI, the same probability of each random UMI can be assumed.

### 2.8.2. Probability Based Merging

2.8.2.1. Family Merging from the Same Strand

After an initial round of family grouping with the same start-end or mismatch ≤1, UMI sequence, and strand, additional grouping is performed on existing families or reads with N base in sequence. For each family/read-pairs, the soft-clip adjusted start-end and strand can be used as group key. For all potential families of the same group key, the user can iterate over any pairs of families to compute the relative likelihood that Families A and B originate from the same family versus different families, “L=P(A=B)/P(A!=B)”. The Likelihood can be calculate by using UMI and positional information, as described below. See FIG. 32 for a graphical illustration.

### 2.8.2.1.1. UMI Information

If UMI are random UMI, for each pair of UMI as s1, s2 from family1 and family2, calculate hamming distance as: Dis, total number of non-match base after excluding N; nN, total number N base in either of the item. Compute probability as follows: set “seq_er” as “sequencing error=0.001”; “P(dis,UMI_length−nN)=0.75\hat{ }dis*0.25\hat{ }(UMI_length−nN-dis)*choose (dis, UMI_length−nN)”; P(A=B): “min(1, seq_er\hat{ }dis+P(jumping prob)*P(dis, UMI_length−nN))”; P(A!=B): “P(dis, UMI_length−nN); Lᵤₘᵢ=P(A=B)/P(A!=B)”.

If UMI are non-random UMI, for each pair of UMI as s1, s2 from family1 and family2. If s1 and s2 are designed UMI, correct as s1′ and s2′ (if the distance between observed and corrected UMI>1, discard the read-pair). For s1 transit to s2, there are, in some embodiments, two possibilities: set “seq_er” as “sequencing error=0.001”; calculate hamming distance between s1 and s2 as “dis1”; calculate sum of hamming distance between s1-s1′ and s2-s2′ as “dis2”; P(A=B): “min(1, seq_er\hat{ }dis1+P(jumping prob)*seq_er\hat{ }dis2); (1) If s1′ equal s2′, P(A!=B): “P(UMI frequency)”; (2) If s1′ not equal to s2′, P(A!=B): 1; “Lᵤₘᵢ=P(A=B)/P(A!=B)”.

### 2.8.2.1.2. Positional Information

Assume the number of unique molecules follows a binomial distribution~binomial (number of candidate, P(insert_size)). Number of candidate=max (number of candidate with same start and strand, number of candidate with same end and strand).

Compute probabilities as follows: P(A=B), “Pr(Number of molecules=1)” and P(A!=B), “Pr(Number of molecules>1)”.

If the positions for Family A and Family B do not exactly match, the probability for fuzzy window can be computed as follows: set indel error as “Indel_ER=0.001” and compute number of fragment ends that are different between Family A and Family B as “frag_end_diff_n”. If Family A and Family B don't have the exact same match for position, “P(A=B)=P(A=B)*Indel_ER\hat{ }frag_end_diff_n”. The Lₚₒₛ can be computed from “Lₚₒₛᵢₜᵢₒₙ=P(A=B)/P(A!=B)”. The final likelihood L=Lᵤₘᵢ*Lₚₒₛᵢₜᵢₒₙ, if L is above the pre-defined threshold merge Families A and B.

### 2.8.2.1.3. Duplex-Merging

After the simplex reads grouping and merging is completed, duplex collapsing can be performed. For each candidate family, loop through all families within the fuzzy window range. For pairs of families with reverse strand information, the pair-wise likelihood can be computed to find the most likely candidate to merge.

If duplex UMI is used, a pair of family that forms a duplex can look like:
UMI_A1-positive strand-UMI_B1
UMI_B2-negative strand-UMI_A2

If simplex UMI is used, a pair of family that forms a duplex can look like:
UMI_A1—positive strand—NNN
NNN—negative strand—UMI_A2

Similar to family merging from the same strand, likelihood “L=P(A=B)/P(A!=B)” is computed using the UMI and positional information.

### 2.8.2.1.4. UMI Information

If simplex UMI is used, “Lᵤₘᵢ=1”. If duplex UMI is used, the probabilities can be computed as follows: assume the sides of UMI that are different between Family A and Family B are s1 and s2; set “seq_er” as “sequencing error=0.001”; calculate hamming distance between s1 and s2 as “dis1”; P(A=B): “min(1, seq_er\hat{ }dis1+P(jumping prob))”; If s1 equal s2, P(A!=B): “P(UMI frequency)”; if s1′ not equal to s2′, P(A!=B): 1.

The number of unique molecule is assumed to follow a binomial distribution~binomial (number of candidate, P(insert_size)). Number of candidate=max (number of candidate with same start and strand, number of candidate with same end and strand).

Compute probabilities as follows: P(A=B): Pr(Number of molecule=1) and P(A!=B): Pr(Number of molecule>1). If the positions for Family A and B do not exactly match, compute probability for fuzzy window as follows: set indel error as “Indel_ER=0.001”; compute number of fragment ends that are different between Family A and Family B as “frag_end_diff_n”; If Family A and B don't have exact same match for position, “P(A=B)=P(A=B)*Indel_ER\hat{ }frag_end_diff_n”.

The Lₚₒₛ can be computed from “Lₚₒₛᵢₜᵢₒₙ=P(A=B)/P(A!=B)”. The final likelihood “L=Lᵤₘᵢ*Lₚₒₛᵢₜᵢₒₙ”, if L is above the pre-defined threshold merge Family A and B.

### 2.9. Read Collapsing

### 2.9.1. Read Filtering

After family grouping with different types of correction, families will get filtered out prior to collapsing if any of the following conditions are met: (1) Read1 or Read2 have supporting reads less than umi-min-supporting-reads and (2) Family simplex/duplex status is not matching the umi-emit-multiplicity (e.g., if umi-emit-multiplicity-duplex, all simplex families will be discarded). If umi-emit-multiplicity=simplex, all duplex families will be discarded.

### 2.9.2. Read Pileup Construction and Candidate Selection

In some embodiments, a list of collapsers are employed to process families, combining the multiple input read pair information they contain into consensus read pairs. In some embodiments, two types of collapsing can be done: simplex collapsing, where accumulated read pileups are combined into consensus reads on one strand; and cross-family collapsing, where consensus reads are those whose UMIs, orientations, and positions indicate that they are from the same dual-stranded source molecule.

When the family is simplex, the simplex collapsing can proceed using the following steps: (1) Group reads by CIGAR string; (2) Produce pileups for each read group; (3) Order pileups descending by read count, ascending by indel distance; (4) Create a consensus read from the first group e.g., the group with largest read count and lowest distance to reference; (5) save a second candidate if read count of second candidate>read count of first candidate*minRatio (default 0.5).

When the family is duplex, cross-family collapsing can work according to the following steps: (1) Obtain the read group candidates from each strand; (2) Compare the two mates of both strands to find a best matching read group (e.g., compare read1 of positive strand with read2 of negative strand); (a) Exact matched CIGAR hypothesis; (b) matched CIGAR string with less difference from reference; (3) Output one consensus read based on the best matching read group; (4) If no matched CIGAR hypothesis from two strands, the two strands can be reported as two separate simplex families.

### 2.9.3. Consensus Base Generation

Once a best read group (CIGAR hypothesis) is selected, the software will start generating the consensus read pair using all the reads with the same CIGAR hypothesis.

The consensus base can be set according to the following rules. The software can calculate the most frequently observed base and the second most frequently observed base. If there are no bases observed, the consensus base can be set to ‘N’. If only 1 base is observed, the consensus base can be set to observed base. If there are two or more bases observed, the consensus base can be set to the most frequently observed base. If top two bases comprise an equal frequency, the consensus base can be set to the one with higher condensed qscore. If the second most frequent base's “count “MajorityRatio” (default 4/3) is greater than or equal to the winner's count, the consensus can be set to ‘N’. For cross-family merging, only two pileups (e.g., read1 from one strand read2 from the opposite strand) are compared to generate consensus base.

### 2.9.4. Quality Score Computation

To compute a new quality score of consensus base, Fisher's method can be applied to represent higher quality score post collapsing. The Fisher score accumulates a sum of the natural log of the basecall likelihoods, whereas a Max score simply keeps the largest score encountered. The detailed steps are described below.

The software converts the original base's qscore to p-value as the following: “p=10\hat{ }(−q/10)”. The software next calculates chi-squared statistics, X², by combining p-value of all bases that agree with the consensus base at the pileup position as the following: “X²=−2*sum of all ln(p)”. The software can calculate p-value of chi-squared statistics X² as the following: “p-value=chisqr(degree of freedom, double Cv)”, where “degree of freedom=2*number of qscores” and “Cv=the X²” from above. The software can convert the p-value into qscore again for the final condensed qscore as the following: “Q=−10*log 10 p-value”.

### 2.9.5. Assign Consensus Read ONAME

Each of the collapsed reads can be generated based on the following convention: “consensus_read_refID1_pos1_refID2_pos2_orientation”.

Where: refID1, reference ID of read1; pos1, genomic position of read1; refID2, reference ID of read2; pos2, genomic position of read2; orientation, orientation of read1 and read2.

Where: 1, read1 is forward and read2 is reverse, read1 start position≤read2 end position; 2, read2 is forward and read1 is reverse, read2 start position≤read1 end position; 3, read1 is forward and read2 is reverse, read1 start position>read2 end position; 4, read2 is forward and read1 is reverse, read2 start position>read1 end position; 5, both read1 and read2 are forward; 6, both read1 and read2 are reverse. Note that in all of these cases, “position” actually refers to the outermost aligned position of the read, adjusted for soft clips.

### 2.10. Realigning Collapsed Reads

As described above, “ReadCollapserThreads” feeds series of “CollapsedRegions” into the “RegionSerializerThread”, which puts the output reads into the expected order and pushes them downstream into the DRAGEN aligner, and from there into the rest of the DRAGEN pipeline. In the initial implementation of this system, it was observed that speed was, in some embodiments, limited by the performance of the memory allocator. The “FamilyHashtable” and “ReadCollapser” logic both hammered the allocator to build data structures and to construct output reads. The “RegionSerializerThread” hammered the allocator with millions of calls to free memory. This performance bottleneck was mitigated by giving each “CollapsedRegion” its own “SingleUseAllocator” object. These allocators get large chunks of memory and hand out small portions to clients without requiring any free( ) calls. Later, when that entire “CollapsedRegion” is complete, all of the memory is released in one large free. By eliminating lock contention between allocation and free, this major speed limitation is relieved.

### Unique Molecular Identifiers

The DRAGEN pipeline can process data from whole genome and hybrid-capture assays with unique molecular identifiers (UMI). UMIs are molecular tags added to DNA fragments before amplification to determine the original input DNA molecule of the amplified fragments. UMIs help reduce errors and biases introduced by DNA damage such as deamination before library prep, PCR error, or sequencing errors.

In some embodiments, to use the UMI Pipeline, the input reads files must be from a paired-end run. Input can be pairs of FASTQ files or aligned/unaligned BAM input. DRAGEN can support the following UMI types: Dual, nonrandom UMIs, such as TruSight Oncology (TSO) UMI Reagents or IDT xGen Prism; Dual, random UMIs, such as Agilent SureSelect XT HS2 molecular barcodes (MBC) or IDT xGen Duplex Seq Adapters; Single-ended, random UMIs, such as Agilent SureSelect XT HS molecular barcodes (MBC) or IDT xGen dual index UMI Adapters.

DRAGEN uses the UMI sequence to group the read pairs by their original input fragment and generates a consensus read pair for each such group, or family. The consensus reduces error rates to detect rare and low frequency somatic variants in DNA samples with high accuracy. The DRAGEN pipeline can generate a consensus as follows: (1) Aligns reads; (2) Groups reads into groups with matching UMI and pair alignments (these groups are referred to as families); (3) Generates a single consensus read pair for each read family. These generated reads have higher quality scores than the input reads and reflect the increased confidence gained by combining multiple observations into each base call. In some embodiments, the UMI workflow is only compatible with small variant calling and SV in DRAGEN.

### UMI Input

UMIs can be entered in any one of the following formats: (1) Read name—The UMI sequence is located in the eighth colon-delimited field of the read name (QNAME), for example, “NDX550136:7:H2MTNBDXX:1:13302:3141:10799:AAGGATG+TCGGAGA”; (2) BAM tag—The UMI is present as an RX tag in pre-aligned or aligned BAM file (standard SAM format) or; (3) FASTQ file—The UMI is located in a third FASTQ file using the same read order as the read pairs. To create FASTQ, the user can append the UMI to the read name, and then specify the appropriate “OverrideCycles” setting in the BCL conversion tool. DRAGEN supports UMIs with two parts each with a maximum of 8 bp and separated by +, or a single UMI with a maximum of 15 bp.

In some embodiments, the UMI workflow must be executed using a set of reads that correspond to a unique set of Read Group Sample Name (RGSM)/Read Group Library (RGLB). DRAGEN supports multiple lanes if all lanes correspond to the same RGSM/RGLB set.

In some embodiments, DRAGEN UMI does not support a tumor-normal analysis, because a tumor-normal run corresponds to two different RGSM. In a tumor-normal run, one sample name can be used for tumor and one sample name can be used for normal. In some embodiments, DRAGEN UMI supports one sample in a run.

If using a BAM file or a list of FASTQ files as the input, the input can contain multiple samples. DRAGEN checks if only one sample is included in the run and if the sample uses only a single, unique RGLB library. DRAGEN also accepts a library that was spread across multiple lanes. If there is a single sample and single library, DRAGEN processes all included reads. If there are multiple samples or multiple libraries, DRAGEN aborts analysis with an error.

### UMI Input Correction Table

For dual, nonrandom UMIs, the user can provide a predefined UMI correction table or a list of valid UMI sequences as input. To create the UMI correction table, use a tab-delimited file, include a header, and add the following fields shown in Table 5.

TABLE 5
UMI INPUT CORRECTION

| Field | Value |
|---|---|
| UMI | The UMI sequence. For example, ACGTAC |
| IsValid | Specify if the UMI sequence is valid. Enter either: |
| | TRUE or FALSE |
| NearestCodes | Colon-separated list of nearest UMI sequences. |
| | For example, ACGTAA:ACGTAT |
| SecondNearestCodes | Colon-separated list of second nearest sequences. |
| | For example, ACGGAA:ACGGAT |

If a customized correction table is not specified, DRAGEN uses the default table for TruSight Oncology (TSO) UMI Reagents located at src/config/umi_correction_table.txt. Alternatively, the user can provide a file for whitelisted nonrandom UMI with valid UMI sequence, one per line. DRAGEN then autogenerates a UMI correction table with hamming distance of one.

### UMI Options

--umi-library-type

The user can set the batch option for different UMIs correction. Three batch modes are available that optimize collapsing configurations for different UMI types. Use one of the following modes:

random-duplex

Dual, random UMIs.

random-simplex

Single-ended, random UMIs.

nonrandom-duplex

Dual, nonrandom UMIs. To use this option, the user can provide a target manifest file using “--umi-metrics-interval-file”.

--umi-min-supporting-reads

The user can specify the number of matching UMI inputs reads required to generate a consensus read. In some embodiments, any family with insufficient supporting reads is discarded. For example, the following are the recommended settings for FFPE and ctDNA: [FFPE] If the variant >1%, use “--umi-min-supporting-reads=1” with the “--vc-enable-umi-solid” variant caller parameter; [ctDNA] If the variant <1%, use “--umi-min-supporting-reads=2” with the “--vc-enable-umi-liquid” variant caller parameter.

--umi-enable

To enable read collapsing, the user can set the “--umi-enable” option to “true”. In some embodiments, this option is not compatible with “-enable-duplicate-marking” because the UMI pipeline generates a consensus read from a set of candidate input reads, rather than choosing the best nonduplicate read. If using the “--umi-library-type” option, “--umi-enable” is not required. umi-emit-multiplicity

The user can set the consensus sequence type to output. DRAGEN UMI allows users to collapse duplex sequences from the two strands of the original molecules. In some embodiments, duplex sequence is typically ~20-60% of total library, depending on library kit, input material, and sequencing depth. The user can enter one of the following consensus sequence types:

### Both

Output both simplex and duplex sequences. This option is the default.

### Simplex

Output only simplex sequences.

### Duplex

Output only duplex sequences.

--umi-source

The user can specify the input type for the UMI sequence. The following are valid values: qname, bamtag, and fastq. If using “--umi-source-fastq”, the UMI sequence from FASTQ file using “--umi-fastq” can be provided.

--umi-correction-table

The user can enter the path to a customized correction table. By default, Local Run Manager uses lookup correction with a built-in table for the TruSight Oncology and for IDT UMI Index Anchor kits from Illumina, Inc.

--umi-nonrandom-whitelist

The user can enter the path for a customized, valid UMI sequence.

--umi-metrics-interval-file

The user can enter the path for target region in BED format.

### Nonrandom and Random UMI Correction

In some embodiments, DRAGEN processes UMIs by grouping reads by UMI and alignment position. If there are sequencing errors in the UMIs, DRAGEN can correct and detect small sequencing errors by using a lookup table or by using sequence similarity and read counts. The user can specify the type of correction with the “--umi-library-type” or “--umi-correction-scheme” option using the values “lookup”, “random”, or “none”.

For sparse sets of nonrandom UMIs, a lookup table can be created that specifies which sequence can be corrected and how to correct it. In some embodiments, this correct file scheme works best on UMI sets where sequences have a minimum hamming/edit distance between them. By default, DRAGEN uses lookup correction with a built-in correction table for the TruSight Oncology and for IDT UMI Index Anchor kits from Illumina, Inc. The user can specify the path of their correction file using the “--umi-correction-table” option. In some embodiments, the user can employ a different set of nonrandom UMIs.

In the random UMI correction scheme, the DRAGEN pipeline, in some embodiments, must infer which UMIs at a given position are likely to be errors relative to other UMIs observed at the same position. The error modes include small UMI errors, such as one mismatch, or UMI jumping or hopping artifact from library prep. DRAGEN accomplishes this as described below.

Reads are grouped by fragment alignment position. Within a small fuzzy window at each position (e.g., 1, 2, 3, 4, or 5), the reads are grouped first by exact UMI sequence, which forms a family. UMI jumping or hopping probability is estimated through insert size distribution and number of distinct UMI at certain positions. Within a fuzzy window, pair-wise likelihood ratio is calculated to assess if two families with different UMI sequences and genomic positions are derived from the same original molecule. Families with likelihood lower than threshold are merged. The default threshold is 1, for example.

### Merge Duplex UMIs

Duplex UMI adapters simultaneously tag both strands of double-stranded DNA fragments. It is then possible to identify reads resulting from amplification of each strand of the original fragment.

In some embodiments, DRAGEN considers two collapsed read pairs to be the sequence of two strands of the same original fragment of DNA if they have the same alignment position (within a fuzzy window), complementary orientations, and their UMIs are swapped from Read 1 and Read 2. If there is only single-ended UMI, DRAGEN compares the start-end position of families from two strands and computes pair-wise likelihood to determine if they likely originated from two distinct families or should be merged as a duplex sequence. By default, DRAGEN outputs both simplex and duplex consensus sequences.

--umi-emit-multiplicity

can be used to change the consensus sequence output type.

### Example UMI Commands

Generate consensus BAM from FASTO

The following is an example DRAGEN command for generating a consensus BAM file from input reads with Illumina UMIs:

dragen \
-r <REF> \
-1 <FQ1> \
-2 <FQ2> \
--output-dir <OUTPUT> \
--output-file-prefix <PREFIX> \
--enable-map-align true \
--enable-sort true \
--umi-library-type nonrandom-duplex \
--umi-metrics-interval-file <valid target BED file>

### Use FASTO UMI Input

To run with other random UMI library type, change

```
--umi-library-type to random-simplex or random-duplex.
  dragen \
  -r <REF> \
  -1 <FQ1> \
  -2 <FQ3> \
  --umi-source=fastq \
  --umi-fastq <FQ2> \
  --output-dir <OUTPUT> \
  --output-file-prefix <PREFIX> \
  --enable-map-align true \
  --enable-sort true \
  --umi-library-type nonrandom-duplex \
  -umi-metrics-interval-file [valid target BED file]
```

### Use Customized Correction Table

dragen \
-r <REF> \
-1 <FQ1> \
-2 <FQ2> \
--umi-correction-table <valid umi correction table>
--output-dir <OUTPUT> \
--output-file-prefix <PREFIX> \
--enable-map-align true \
--enable-sort true \
--umi-library-type nonrandom-duplex \
--umi-metrics-interval-file <valid target BED file>

### UMI Outputs

### Collapsed BAM

If the user enables BAM output, DRAGEN generates an “<output_prefix>.bam” that includes all UMI consensus reads. The QNAMEs for the reads are generated based on the following convention:
consensus_read_refID1_pos1_refID2_pos2_orientation

Where: refID1, the reference ID of Read 1; pos1, the genomic position of Read 1; refID2, the reference ID of Read 2; pos2, the genomic position of Read 2; orientation, The orientation of Read 1 and Read 2.

Orientation can be one of the following values (Position refers to the outermost aligned position of the read and is adjusted for soft clips): 1, Read 1 is forward and Read 2 is reverse, the starting position for Read 1 is less than or equal to the Read 2 end position; 2, Read 1 is reverse and Read 2 is forward, the starting position for Read 2 is greater than or equal to the Read 1 end position; 3, Read 1 is forward and Read 2 is reverse, the starting position for Read 1 is greater than the Read 2 end position; 4, Read 1 is reverse and Read 2 is forward, the starting position for Read 2 is greater than the Read 1 end position; 5, Read 1 and Read 2 are forward; and 6, Read 1 and Read 2 are reverse.

### UMI Metrics

DRAGEN outputs an “<output_prefix>.umi_metrics.csv” file that describes the statistics for UMI collapsing. This file summarizes statistics on input reads, how they were grouped into families, how UMIs were corrected, and how families generated consensus reads. The following metrics described below can be useful when tuning the pipeline for an application.

### Discarded Families

Any families having fewer than “--umi-min-supporting-reads” input or having a different duplex/simplex status than specified by

--umi-emit-multiplicity
can be discarded. These reads can be logged as Reads filtered out. The families can be logged as Families discarded.

### UMI Correction

Families can be combined in various ways. The number of such corrections can be reported as follows: (1) Families shifted, where families with fragment alignment coordinates up to the distance specified by the

umi-fuzzy-window-size
parameter are merged. The default “umi-fuzzy-window-size” parameter is 3; (2) Families contextually corrected, where families with exactly the same fragment alignment coordinates and compatible UMIs are merged; or (3) Duplex families, where families with close alignment coordinates and complementary UMIs are merged.

When the user specifies a valid path for “--umi-metrics-interval-file”, DRAGEN outputs a separate set of on-target UMI statistics that contains only families within the specified BED file.

If the user needs to analyze the extent to which the observed UMIs cover the full space of possible UMI sequences, the histogram of unique UMIs per fragment position metric may be helpful. It is a zero-based histogram, where the index indicates a count of unique UMIs at a particular fragment position and the value represents the number of positions with that count.

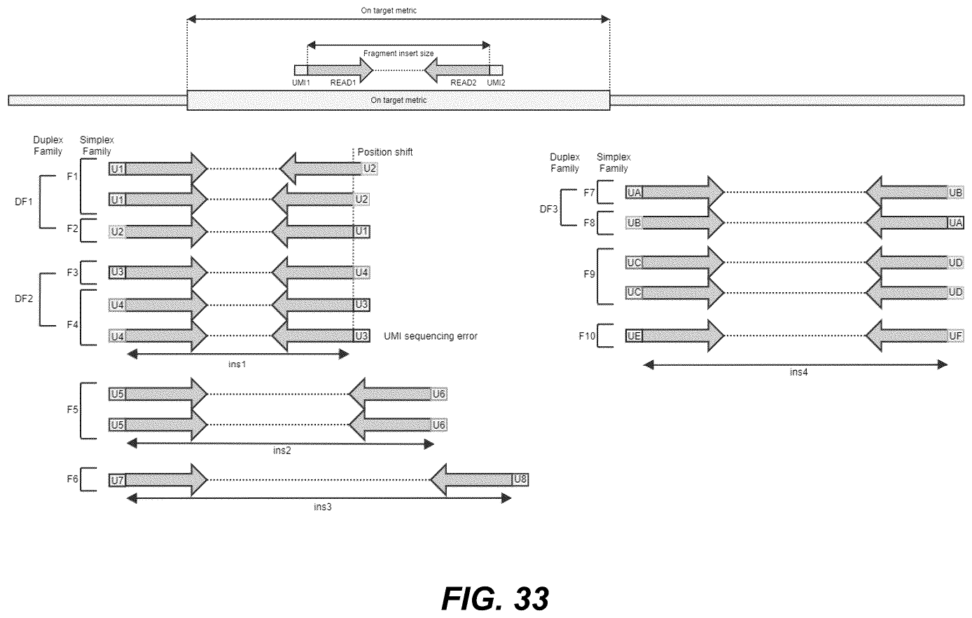

Table 6 below and FIG. 33-FIG. 35 describe non-limiting examples of available UMI metrics.

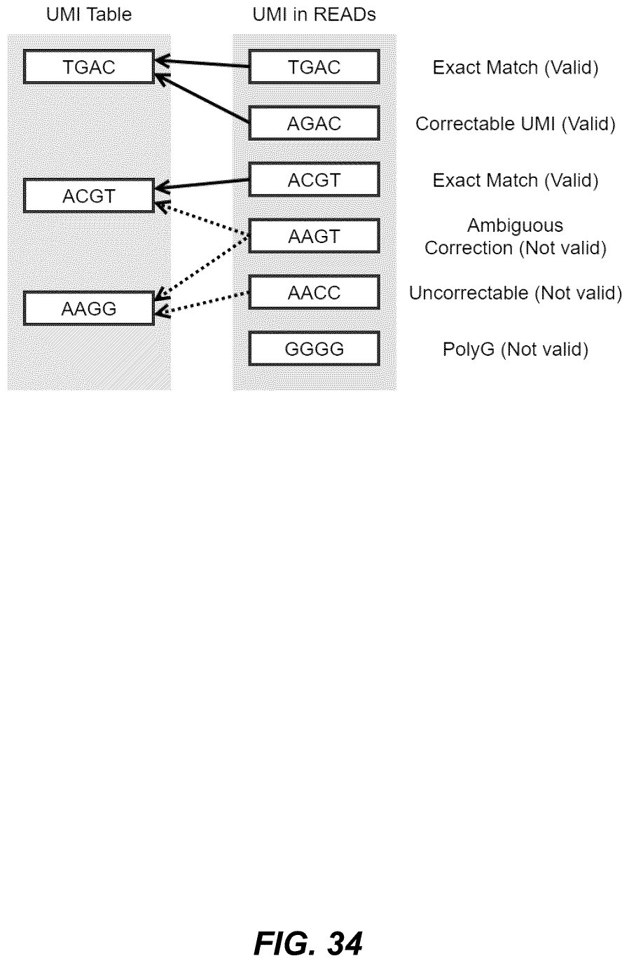

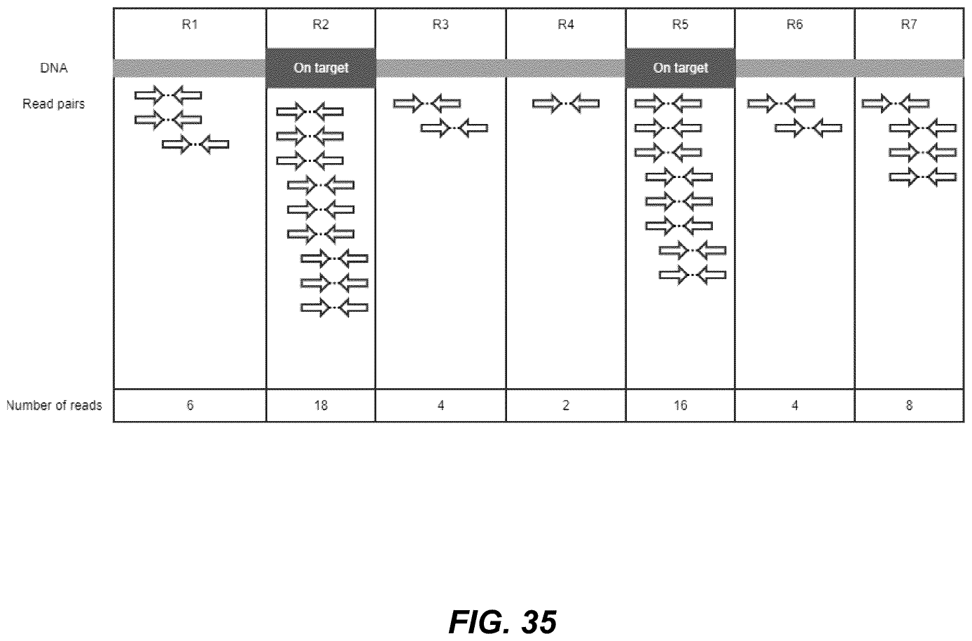

TABLE 6
UMI Metrics

| | | Denominator of | |
|---|---|---|---|
| Metric | Description | percentile | Example |
| Number of reads | Total number of reads. | NA | FIG. 33, 14 pairs of read X |
| | | | 28 = reads |
| Number of reads with | Number of reads for | Number of reads | FIG. 34, Valid UMI read |
| valid or correctable | which the UMIs can be | | count (Exact match + |
| UMIs | corrected based on the | | Correctable UMI) |
| | lookup table. | | |
| Number of | Number of reads in | Number of reads | FIG. 33, Number of reads in |
| reads in | discarded families. | | Families discarded (See |
| discarded | Families are discarded | | “Families discarded” for |
| families | when there are not | | more detail) |
| | enough raw reads to | | |
| | support the family | | |
| | (family size less than | | |
| | “--umi-min-supporting- | | |
| | reads”). In some | | |
| | embodiments, For | | |
| | “--umi-emit- | | |
| | multiplicity = duplex” | | |
| | option, simplex families | | |
| | will be discarded. | | |
| Reads filtered out | Number of reads filtered | Number of reads | Number of reads in |
| | out in total, either for | | discarded families + Reads |
| | properties or in a | | with all-G UMI+ Number of |
| | discarded family. | | unpaired reads |
| Reads with all-G UMIs | Number of reads filtered | Number of reads | FIG. 34, PolyG UMI read |
| filtered out | out due to all-G in UMI | | count |
| | sequence. | | |
| Reads with | Number of reads where | Number of reads | FIG. 34, Uncorrectable + |
| uncorrectable UMIs | the UMI could not be | | Ambiguous correction + |
| | corrected. | | PolyG |
| Total number of families | Number of simplex | NA | FIG 33, F1~F10. |
| | collapsed reads | | |
| Families contextually | Contextual correction is | Total number of | FIG. 34, Family count of |
| corrected | based on other families | families | correctable UMI |
| | at the same mapping | | |
| | location including UMI | | |
| | sequencing error and | | |
| | UMI jumping. | | |
| Families shifted | Number of families that | Total number of | FIG. 33, First read pair of |
| | have some shift | families | DF1 (shifted distance ≤ |
| | correction. Shift | | “umi-fu window-size”) |
| | correction merges | | |
| | families with fragment | | |
| | alignment coordinates | | |
| | up to the distance | | |
| | specified by the umi- | | |
| | fuzzy-window- | | |
| | size parameter. | | |
| Families discarded | Number of families | Total number of | FIG. 33, Families discarded |
| | filtered out by failing | families | by support-reads + Families |
| | min supporting reads | | discarded by duplex/simple |
| | criteria or umi-emit type | | (See below for detail) |
| | of simplex/duplex. | | |
| Families discarded by | Number of families | Total number of | FIG. 33, Number of families |
| min-support-reads | filtered out by failing | families | size less than “umi-min- |
| | minimum supporting | | support reads” option |
| | reads criteria. | | Size 1: F6, F10 |
| | | | Size 2: DF3, F5, F9 |
| | | | Size 3: DF1, DF2 |
| Families discarded by | Number of families | Total number of | FIG. 33, Number of simplex |
| duplex/simplex | filtered out by failing | families | fa (F5, F6, F9, F10) filtered. |
| | umi-emit type of | | Note that simplex reads are |
| | simplex/duplex. | | filtered if umi-emit- |
| | | | multiplicity = duplex (default: |
| | | | both) |
| Families with | Number of families | Total number of | FIG. 34, Number of families |
| ambiguous correction | where the UMI cannot | families | of ambiguous correction |
| | be corrected because | | UMI |
| | more than one possible | | |
| | UMI correction exists. | | |
| Duplex families | Number of families that | Consensus pairs | FIG. 33, DF1, DF2, DF3 |
| | are merged as duplex | emitted | |
| | (both strands). | | |
| Consensus pairs emitted | Number of collapsed | NA | FIG. 33, Depends on umi- |
| | reads in output BAM. | | emit |
| | | | multiplicity = simplex/duplex/ |
| | | | umi-min-supporting-- |
| | | | reads = simplex = F1~F10 (F2, |
| | | | F3, F6, F7, F8, F10 filtered |
| | | | if x >= 2) |
| | | | duplex = DF1, DF2, DF3 |
| | | | both = DF1, DF2, DF3, F5, |
| | | | F6, F9, F10 (F6, F10 filtered |
| | | | if x ≥ 2) |
| Mean family depth | Average number of read | NA | FIG. 33, Number of reads |
| | pairs per family. Filtered | | per family: |
| | reads and families are | | DF1 = 3, DF2 = 3, DF3 = 2, |
| | excluded. | | F5 = 2, F6 = 1, F9 = 2, F10 = 1 |
| | | | Mean family depth = |
| | | | (3 + 3 + 2 + 2 + 1 + 2 + 1)/7 = 2 |
| Histogram of num | Number of families with | NA | FIG. 33, |
| supporting fragments | zero raw reads, one raw | | 0 reads: None |
| | read, two raw reads, | | 1 read: F6, F10 = 2 (0 if |
| | three raw reads, etc | | umi-min-supporting- |
| | | | reads = 2) |
| | | | 2 reads: DF3, F5, F9 = 3 |
| | | | 3 reads: DF1, DF2 = 2 |
| | | | Histogram = {0\|0\|3\|2} |
| Number of collapsible | Number of regions. | NA | FIG. 35, R1~R7 |
| regions | | | |
| Min collapsible region | Number of reads in the | NA | FIG. 35, 2 reads (R4) |
| size (num reads) | least populated region. | | |
| Max collapsible region | Number of reads in the | NA | FIG. 35, 18 reads (R2) |
| size (num reads) | most populated region. | | |
| Mean collapsible region | Average number of | NA | FIG. 35, 8.3 |
| size (num reads) | reads per region. | | |
| region size standard | Standard deviation of | NA | FIG. 35, 5.8 |
| deviation | the number of reads per | | |
| | region. | | |
| On target number of | Number of reads that | NA | FIG. 33 and FIG 35, All on |
| reads | overlapped with the | | target metrics are same as |
| | UMI target interval | | corresponding metric but |
| | --umi-metrics- | | only considering fragments |
| | interval-file. | | overlap with target intervals, |
| | | | i.e. DF3, F9, F10 in FIG. 33 |
| | | | and R1, R3, R4, R6, R7 in |
| | | | FIG. 35 excluded from |
| | | | metric |
| On target number of | Number of reads with a | On target number of | |
| reads with valid or | UMI that matched a | reads | |
| correctable UMIs | UMI in the lookup table, | | |
| | including error | | |
| | allowance, and | | |
| | overlapped with the | | |
| | UMI target interval. | | |
| On target number of | Number of reads in | On target number of | |
| reads in discarded | discarded families that | reads | |
| families | overlapped with the | | |
| | UMI target interval. | | |
| On target duplex | Number of families that | On target consensus | |
| families | are merged as duplex | pairs emitted | |
| | among all the families | | |
| | that are overlapped with | | |
| | UMI target interval. | | |
| On target mean family | Average number of | NA | |
| depth | reads per family that | | |
| | overlapped with UMI | | |
| | target interval. | | |
| On target families | Number of families that | On target number of | |
| discarded | overlapped with UMI | families | |
| | target interval filtered | | |
| | out by failing min | | |
| | supporting reads criteria | | |
| | or umi-emit type of | | |
| | simplex/duplex. | | |
| On target families | Number of families that | On target number of | |
| discarded by min- | overlapped with UMI | families | |
| support-reads | target interval filtered | | |
| | out by failing min | | |
| | supporting reads criteria. | | |
| On target families | Number of families that | On target number of | |
| discarded by | overlapped with UMI | families | |
| duplex/simplex | target interval filtered | | |
| | out by failing umi-emit | | |
| | type of simplex/duplex. | | |
| On target families with | Number of families that | On target number of | |
| ambiguous correction | overlapped with UMI | families | |
| | target interval where the | | |
| | UMI cannot be corrected | | |
| | because more than one | | |
| | possible UMI correction | | |
| | exists. | | |
| Histogram of unique | Number of positions | NA | FIG. 33, |
| UMIs per fragment | with zero UMI | | 0 UMI sequence: None. |
| position | sequences, one UMI | | 1 UMI sequences: ins2 (F5), |
| | sequence, two UMI | | ins3 (F6). 2 UMI sequences: |
| | sequences, etc. | | ins1 (DF1, DF2). 3 UMI |
| | | | sequences: ins4 (DF3, F9, |
| | | | F10) Histogram = {0\|2\|1\|1}. |
| Total Families in | Total number of families | NA | |
| Probability Model | used in estimation of | | |
| Estimation | UMI jumping rate and | | |
| | fragment size | | |
| | distribution used for | | |
| | probabilistic family | | |
| | merging. | | |
| Number of potential | Total number of families | Total Families in | |
| Jumping Families | that are potential UMI | Probability Model | |
| | jumping candidates and | Estimation | |
| | the corresponding ratio. | | |

### Grouping Sequence Reads

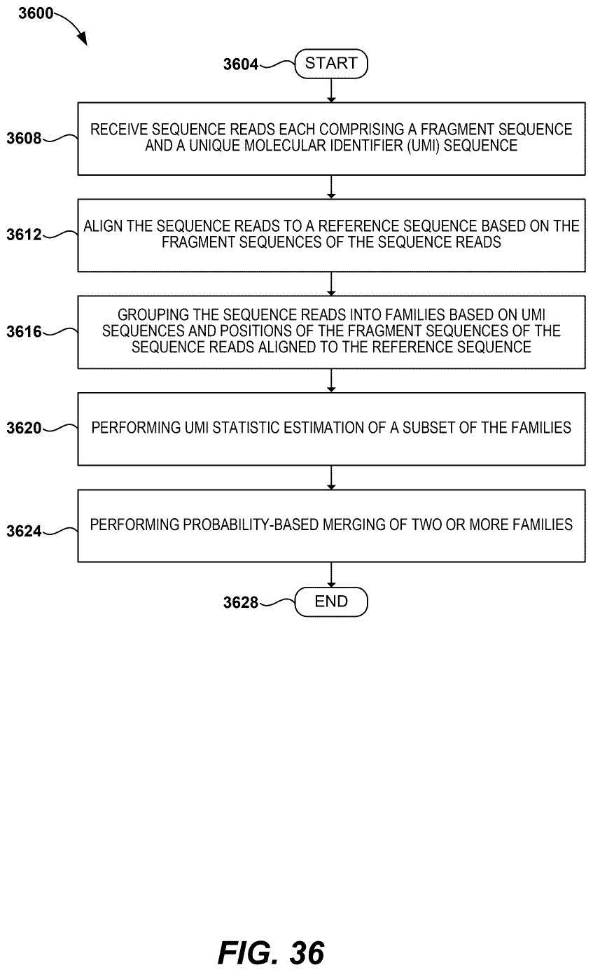

FIG. 36 is a flow diagram showing an exemplary method 3600 of grouping sequence reads. A method of grouping sequence reads can include grouping sequence reads can include grouping sequence reads into families of sequence reads; and merging (or collapsing) families of sequence reads. In some embodiments, reads are grouped by fragment alignment position. Within a small fuzzy window at each position (e.g., 1, 2, 3, 4, or 5), the reads are grouped first by exact UMI sequence, which forms a family. UMI jumping or hopping probability is estimated through insert size distribution and number of distinct UMI at certain positions. Within a fuzzy window, pair-wise likelihood ratio is calculated to assess if two families with different UMI sequences and genomic positions are derived from the same original molecule. Families with likelihood lower than threshold are merged. The default threshold is 1, for example.

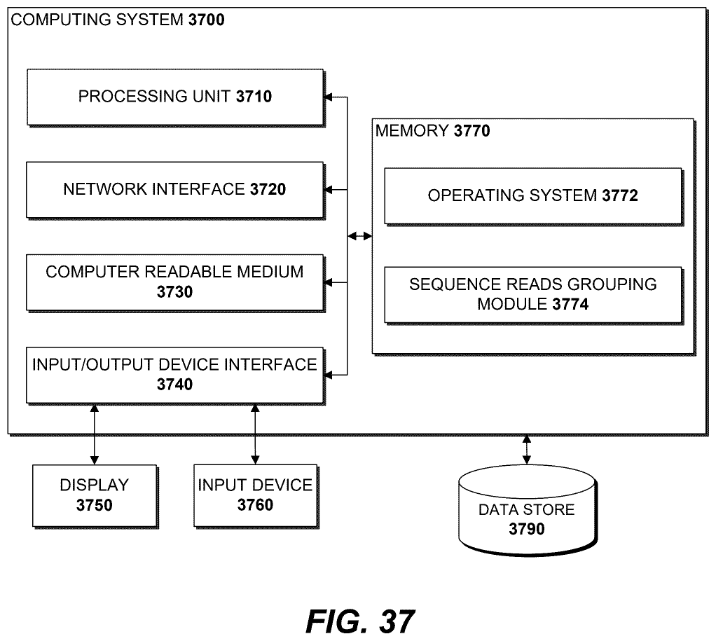

The method 3600 may be embodied in a set of executable program instructions stored on a computer-readable medium, such as one or more disk drives, of a computing system. For example, the computing system 3700 shown in FIG. 37 and described in greater detail below can execute a set of executable program instructions to implement the method 3600. When the method 3600 is initiated, the executable program instructions can be loaded into memory, such as RAM, and executed by one or more processors of the computing system 3700. Although the method 3600 is described with respect to the computing system 3700 shown in FIG. 37, the description is illustrative only and is not intended to be limiting. In some embodiments, the method 3600 or portions thereof may be performed serially or in parallel by multiple computing systems.

After the method 3600 begins at block 3604, the method 3600 proceeds to block 3608, where a computing system (e.g., the computing system 3700 described with reference to FIG. 37) receives a plurality of sequence reads each comprising a fragment sequence and a unique molecular identifier (UMI) sequence (or an identifier sequence). The plurality of sequence reads can be generated from a sample. The sample can be obtained from a subject. The sample can be generated from another sample obtained from a subject. The other sample can be obtained directly from the subject. The sample can comprise cells, cell-free DNA, cell-free fetal DNA, circular tumor DNA, amniotic fluid, a blood sample, a biopsy sample, or a combination thereof. The computing system can load the plurality of sequence reads into its memory. Sequence reads can be generated by techniques such as sequencing by synthesis, sequencing by binding, or sequencing by ligation. Sequence reads can be generated using instruments such as MINISEQ, MISEQ, NEXTSEQ, HISEQ, and NOVASEQ sequencing instruments from Illumina, Inc. (San Diego, CA).

A sequence read can be, for example, 50, 60, 70, 80, 90, 100, 110, 120, 130, 140, 150, 160, 170, 180, 190, 200, 300, 400, 500, 600, 700, 800, 900, 1000, 1250, 1500, 1750, 2000, or more base pairs (bps) in length. For example, a sequence read are about 50 base pairs to about 500 base pairs in length. The sequence reads can comprise paired-end sequence reads. The sequence reads can comprise single-end sequence reads. The sequence reads can be generated by whole genome sequencing (WGS). The WGS can be clinical WGS (cWGS). The sequence reads can comprise single-end sequence reads. The plurality of sequence reads can be generated by whole genome sequencing (WGS), e.g., clinical WGS (cWGS). The sequence reads can be generated by targeted sequencing, such as sequencing of 5, 10, 20, 30, 40, 50, 100, 200, or more genes. The sample can comprise cells, cell-free DNA, cell-free fetal DNA, amniotic fluid, a blood sample, a biopsy sample, or a combination thereof.

A sequence read can include one UMI sequence. A sequence read can comprise two UMI sequences (e.g., a first UMI sequence and a second UMI sequence). The first UMI sequence can be 5′ to the fragment sequence. The second UMI sequence can be 3′ to the fragment sequence. Alternatively, the first UMI sequence can be 3′ to the fragment sequence. The second UMI sequence can be 5′ to the fragment sequence. The first UMI sequence and the second UMI sequence can have different lengths. The first UMI sequence and the second UMI sequence can have an identical length. The first UMI sequence and the second UMI sequence can be different. The first UMI sequence and the second UMI sequence can be identical. A UMI sequence can be, for example, 3, 4, 5, 6, 7, 8, 9, 10, 11, 12, 13, 14, 15, 16, 17, 18, 19, 20, 30, 40, 50 or more or less bases in length. The UMI sequences can be random. The UMI sequences can be non-random.

The method 3600 proceeds from block 3608 to block 3612, where the computing system aligns sequence reads of the plurality of sequence reads to a reference sequence using the fragment sequences of the sequence reads. The reference sequence can be a reference genome sequence (e.g., hg38 or hg19, or a portion thereof). The computing system can align sequence reads to the reference sequence using an aligner or an alignment method such as Burrows-Wheeler Aligner (BWA), ISAAC, BarraCUDA, BFAST, BLASTN, BLAT, Bowtie, CASHX, Cloudburst, CUDA-EC, CUSHAW, CUSHAW2, CUSHAW2-GPU, drFAST, ELAND, ERNE, GNUMAP, GEM, GensearchNGS, GMAP and GSNAP, Geneious Assembler, LAST, MAQ, mrFAST and mrsFAST, MOM, MOSAIK, MPscan, Novoaligh & NovoalignCS, NextGENe, Omixon, PALMapper, Partek, PASS, PerM, PRIMEX, QPalma, RazerS, REAL, cREAL, RMAP, rNA, RT Investigator, Segemehl, SeqMap, Shrec, SHRIMP, SLIDER, SOAP, SOAP2, SOAP3 and SOAP3-dp, SOCS, SSAHA and SSAHA2, Stampy, STORM, Subread and Subjunc, Taipan, UGENE, VelociMapper, XpressAlign, and ZOOM.

The method 3600 proceeds from block 3612 to block 3616, where the computing system groups sequence reads of the plurality of sequence reads into a plurality of families of sequence reads based on the UMI sequences and/or positions of the fragment sequences of the sequence reads aligned to the reference sequence. A family can comprise one sequence read. A family can comprise at least 2 sequence read (e.g., at least 2, 3, 4, 5, 10, 15, 20, 30, 40, 50, 60, 70, 80, 90, 100, 250, 500, 1000, 2000, or more or less sequence reads) of the plurality of sequence reads. A family can comprise sequence reads with an identical UMI sequence, an identical alignment position (referred to herein as exact same start-end), and an identical strand (referred to herein as same strand, e.g., plus strand or minus strand). A family can comprise two sequence reads with an identical UMI sequence, alignment positions that differ within a fuzzy window (e.g., alignment positions can differ by one position (referred to herein as mismatch≤1)), and an identical strand orientation (referred to herein as same strand, e.g., plus strand or minus strand). A fuzzy window can be, for example, 1, 2, 3, 4, or 5. The plurality of families can comprise, for example, at least 100,000, 200,000, 300,000, 400,000, 500,000, 1,000,000, 2,000,000, 3,000,000, 4,000,000, 5,000,000, 10,000,000, or more or less families.

The method 3600 proceeds from block 3616 to block 3620, where the computing system performs UMI statistic estimation of the plurality of families. To perform UMI statistic estimation, the computing system can determine fragment (or fragment insert) size frequency, UMI jumping rate, and/or UMI frequency. See section 2.8 above for an illustration.

The computing system can perform UMI statistic estimation on a subset of families of the plurality of families. The subset of families can comprise at least 5,000, 10,000, 20,000, 30,000, 40,000, 50,000 60,000, 70,000, 80,000, 90,000, 100,000, or more or less, families of the plurality of families. The subset of families can comprise at least 0.1%, 0.5%, 1%, 2%, 3%, 4%, 5%, 6%, 7%, 8%, 9%, 10%, 11%, 12%, 13%, 14%, 15%, 20%, or more or less, of families of the plurality of families.

The method 3600 proceeds from block 3620 to block 3624, where the computing system performs probability-based merging of families of the plurality of families (also referred to herein as read or UMI grouping or collapsing). See section 2.9 above for an illustration. To perform probability-based merging, the computing system can perform family identification and merging (or collapsing). The computing system can perform duplex identification and merging (or collapsing). See FIG. 2 and accompanying description. The computing system can perform probability-based merging of families of the plurality of families using a probability map (see FIG. 12 and the accompanying description for an illustration). After the probability-based merging, the plurality of families can comprise, for example, at least 100,000, 200,000, 300,000, 400,000, 500,000, 1,000,000, 2,000,000, 3,000,000, 4,000,000, 5,000,000, 10,000,000, or more or less families. The plurality of families before probability-based merging is performed can comprise at least 0.5%, 1%, 2%, 3%, 4%, 5%, 6%, 7%, 8%, 9%, 10%, 15%, 20%, 25%, 30%, more families than the plurality of families after probability-based merging is performed. A family after probability-based merging can comprise one sequence read. A family after probability-based merging can comprise at least 2 sequence read (e.g., at least 2, 3, 4, 5, 10, 15, 20, 30, 40, 50, 60, 70, 80, 90, 100, 250, 500, 1000, 2000, or more or less sequence reads) of the plurality of sequence reads.

The computing system can perform probability-based merging of families of the plurality of families using the results of UMI statistic estimation (e.g., fragment size frequency, UMI jumping rate, and/or UMI frequency). The computing system can perform probability-based merging of families of the plurality of families using fragment size frequency, UMI jumping rate, and/or UMI frequency. The computing system can perform probability-based merging of families of the plurality of families using a sequencing error rate (e.g., 0.0001, 0.0002, 0.0003, 0.0004, 0.0005, 0.0006, 0.0007, 0.0008, 0.0009, 0.001, 0.002, 0.003, 0.004, 0.005, or more or less) and/or a mismatch probability (e.g., 0.15, 0.17, 0.2, 0.23, 0.24, 0.25, 0.26, 0.27, 0.3, 0.33, 0.35, or more or less). The sequencing error rate can be predetermined. The mismatch probability can be predetermined.

To perform probability-based merging, the computing system can determine a relative likelihood (or probability) (also referred to herein as L) of the two families are derived from (or that originate from) the same original nucleic acid (e.g., DNA) molecule. The computing system can determine the relative likelihood of the two families are derived from the same original nucleic acid molecule using P(C1-C2) and P(C1!=C2) (see equation 1 and Table 1 for details). The computing system can determine the relative likelihood of the two families are derived from the same original nucleic acid molecule using one or more of equations 4 to 11. The computing system can determine the relative likelihood of the two families are derived from the same original nucleic acid molecule using the fragment size frequency, the UMI jumping rate, and/or the UMI frequency. The computing system can determine the relative likelihood is above a merging threshold (e.g., 1, 1.1, 1.2, 1.3, 1.4, 1.5, 1.6, 1.7, 1.8, 1.9, 2, 3, 4, 5, 6, 7, 8, 9, 10, or more or less)). The computing system can merge the two families of the plurality of families. The computing system can merge a smaller family (e.g., with fewer sequence reads) of the two families into a larger family (e.g., with more sequence reads) of the two families.

To determine the relative likelihood of the two families are derived from the same original nucleic acid molecule, the computing system can determine a likelihood ratio of unique molecule (or family) over non-unique molecule (or family) given fragment positions (also referred to herein as Lₚₒₛ). The computing system can determine a likelihood ratio of UMI transition for unique molecule (or family) over non-unique molecule (or family). This likelihood ratio of UMI transition is referred to herein as Lᵤₘᵢ. UMI transition can be a result of UMI jumping or sequencing error. The computing system can determine the relative likelihood of the two families are derived from the same original nucleic acid molecule as a product (e.g., multiplication product) of (i) the likelihood (or probability) ratio of unique molecule over non-unique molecule given fragment positions and (ii) the likelihood (or probability) ratio of UMI transition for unique molecule over non-unique molecule. The computing system can determine relative likelihood of the two families are derived from the same original nucleic acid molecule using a sequencing error rate and/or a mismatch probability.

To perform probability-based merging, the computing system can (i) for one, one or more, or each pair of families of the plurality of families, determine a relative likelihood (or probability) of the families of the pair are derived from the same original nucleic acid molecule. The computing system can (ii) for the pair of families with the highest relative likelihood (or probability), if the relative likelihood of the families in the pair with the highest relative likelihood (or probability) are derived from the same original nucleic acid molecule is above a merging threshold (e.g., 1), then merging the families. In some embodiments, the computing system can (iii) repeat (i) and (ii) until the relative likelihood of the families in the pair with the highest relative likelihood (or probability) is not above the merging threshold.

The computing system can align the consensus fragment sequence to the reference sequence. In some embodiments, the computing system can determine a fragment sequence and/or a UMI sequence of the original nucleic acid molecule from which the sequence reads of the family are derived. The fragment sequence of the original nucleic acid molecule from which the sequence reads of the family are derived can be a consensus fragment sequence of the family. The UMI sequence of the original nucleic acid molecule from which the sequence reads of the family are derived can be a consensus UMI sequence of the family. The computing system can align the fragment sequence to the reference sequence.

In some embodiments, computing system can create a file or a report and/or generate a user interface (UI) comprising a UI element representing or comprising, for one, one or more, or each of the plurality of families, (i) the family. The file or report and/or the UI element can represent or comprise (ii) sequence reads of the family, fragment sequences of the family, and/or UMI sequences of the family. The file or report and/or the UI element can represents or comprise (iii) a consensus fragment sequence of the family, a position of the consensus fragment sequence aligned to the reference sequence, and/or a consensus UMI sequence of the family. A UI element can be a window (e.g., a container window, browser window, text terminal, child window, or message window), a menu (e.g., a menu bar, context menu, or menu extra), an icon, or a tab. A UI element can be for input control (e.g., a checkbox, radio button, dropdown list, list box, button, toggle, text field, or date field). A UI element can be navigational (e.g., a breadcrumb, slider, search field, pagination, slider, tag, icon). A UI element can informational (e.g., a tooltip, icon, progress bar, notification, message box, or modal window). A UI element can be a container (e.g., an accordion).

The method 3600 ends at block 3628.

### Execution Environment

FIG. 37 depicts a general architecture of an example computing device 3700 configured to execute the processes and implement the features described herein. The general architecture of the computing device 3700 depicted in FIG. 37 includes an arrangement of computer hardware and software components. The computing device 3700 may include many more (or fewer) elements than those shown in FIG. 37. It is not necessary, however, that all of these generally conventional elements be shown in order to provide an enabling disclosure. As illustrated, the computing device 3700 includes a processing unit 3710, a network interface 3720, a computer readable medium drive 3730, an input/output device interface 3740, a display 3750, and an input device 3760, all of which may communicate with one another by way of a communication bus. The network interface 3720 may provide connectivity to one or more networks or computing systems. The processing unit 3710 may thus receive information and instructions from other computing systems or services via a network. The processing unit 3710 may also communicate to and from memory 3770 and further provide output information for an optional display 3750 via the input/output device interface 3740. The input/output device interface 3740 may also accept input from the optional input device 3760, such as a keyboard, mouse, digital pen, microphone, touch screen, gesture recognition system, voice recognition system, gamepad, accelerometer, gyroscope, or other input device.

The memory 3770 may contain computer program instructions (grouped as modules or components in some embodiments) that the processing unit 3710 executes in order to implement one or more embodiments. The memory 3770 generally includes RAM, ROM and/or other persistent, auxiliary or non-transitory computer-readable media. The memory 3770 may store an operating system 3772 that provides computer program instructions for use by the processing unit 3710 in the general administration and operation of the computing device 3700. The memory 3770 may further include computer program instructions and other information for implementing aspects of the present disclosure.

For example, in one embodiment, the memory 3770 includes a sequence reads grouping module 3774 for grouping sequence reads (which can include merging or collapsing families of sequence reads). The sequence reads grouping module 3774 can perform one or more actions of the method 3600 described with reference to FIG. 36. In addition, memory 3770 may include or communicate with the data store 3790 and/or one or more other data stores that store sequence reads or data being processed and results (e.g., intermediate results or final results) of grouping sequence reads.

### ADDITIONAL CONSIDERATIONS

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

One skilled in the art will appreciate that, for this and other processes and methods disclosed herein, the functions performed in the processes and methods can be implemented in differing order. Furthermore, the outlined steps and operations are only provided as examples, and some of the steps and operations can be optional, combined into fewer steps and operations, or expanded into additional steps and operations without detracting from the essence of the disclosed embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Accordingly, phrases such as “a device configured to” are intended to include one or more recited devices. Such one or more recited devices can also be collectively configured to carry out the stated recitations. For example, “a processor configured to carry out recitations A, B and C can include a first processor configured to carry out recitation A and working in conjunction with a second processor configured to carry out recitations B and C. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

It will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

It is to be understood that not necessarily all objects or advantages may be achieved in accordance with any particular embodiment described herein. Thus, for example, those skilled in the art will recognize that certain embodiments may be configured to operate in a manner that achieves or optimizes one advantage or group of advantages as taught herein without necessarily achieving other objects or advantages as may be taught or suggested herein.

All of the processes described herein may be embodied in, and fully automated via, software code modules executed by a computing system that includes one or more computers or processors. The code modules may be stored in any type of non-transitory computer-readable medium or other computer storage device. Some or all the methods may be embodied in specialized computer hardware.

Many other variations than those described herein will be apparent from this disclosure. For example, depending on the embodiment, certain acts, events, or functions of any of the algorithms described herein can be performed in a different sequence, can be added, merged, or left out altogether (for example, not all described acts or events are necessary for the practice of the algorithms). Moreover, in certain embodiments, acts or events can be performed concurrently, for example through multi-threaded processing, interrupt processing, or multiple processors or processor cores or on other parallel architectures, rather than sequentially. In addition, different tasks or processes can be performed by different machines and/or computing systems that can function together.

The various illustrative logical blocks and modules described in connection with the embodiments disclosed herein can be implemented or performed by a machine, such as a processing unit or processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor can be a microprocessor, but in the alternative, the processor can be a controller, microcontroller, or state machine, combinations of the same, or the like. A processor can include electrical circuitry configured to process computer-executable instructions. In another embodiment, a processor includes an FPGA or other programmable device that performs logic operations without processing computer-executable instructions. A processor can also be implemented as a combination of computing devices, for example a combination of a DSP and a microprocessor, a plurality of microprocessors, one or more microprocessors in conjunction with a DSP core, or any other such configuration. Although described herein primarily with respect to digital technology, a processor may also include primarily analog components. For example, some or all of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

Any process descriptions, elements or blocks in the flow diagrams described herein and/or depicted in the attached figures should be understood as potentially representing modules, segments, or portions of code which include one or more executable instructions for implementing specific logical functions or elements in the process. Alternate implementations are included within the scope of the embodiments described herein in which elements or functions may be deleted, executed out of order from that shown, or discussed, including substantially concurrently or in reverse order, depending on the functionality involved as would be understood by those skilled in the art.

It should be emphasized that many variations and modifications may be made to the above-described embodiments, the elements of which are to be understood as being among other acceptable examples. All such modifications and variations are intended to be included herein within the scope of this disclosure and protected by the following claims.

What is claimed is:

1. A method for determining a sequence of an original nucleic acid molecule, comprising:
preparing a nucleic acid sequencing library from a biological sample, wherein preparing the nucleic acid sequencing library comprises tagging nucleic acid fragments with unique molecular identifier (UMI) sequences to produce UMI-tagged nucleic acid fragments;
sequencing the UMI-tagged nucleic acid fragments using a high-throughput sequencing platform to generate a plurality of sequence reads, wherein each sequence read comprises a fragment sequence and a UMI sequence, and wherein the high-throughput sequencing platform introduces sequencing-platform-specific artifacts including UMI jumping;
under control of a hardware processor:
aligning the plurality of sequence reads to a reference sequence using the fragment sequences of the sequence reads;
grouping the aligned plurality of sequence reads into a plurality of families of sequence reads based on the UMI sequences and positions of the fragment sequences aligned to the reference sequence;
performing UMI statistic estimation on the plurality of families, wherein performing UMI statistic estimation comprises determining, from the plurality of families, one or more of: fragment size frequency, UMI jumping rate, and UMI frequency, wherein the UMI statistic estimation characterizes sequencing-platform-specific artifacts present in the plurality of families;
performing probability-based merging of families of the plurality of families using results of the UMI statistic estimation to correct for the sequencing-platform-specific artifacts, wherein performing probability-based merging comprises:
determining a relative likelihood that a first family and a second family of the plurality of families are derived from the same original nucleic acid molecule, wherein the relative likelihood is a product of:
a likelihood ratio of a unique molecule over a non-unique molecule given the fragment positions of the first family and the second family, and
a likelihood ratio of UMI transition for a unique molecule over a non-unique molecule;
determining that the relative likelihood exceeds a merging threshold; and
merging the first family and the second family into a merged family in response to determining that the relative likelihood exceeds the merging threshold;
subsequent to performing probability-based merging, determining a consensus fragment sequence for each of one or more of the plurality of families; and
determining the sequence of the original nucleic acid molecule based on the consensus fragment sequences,
wherein the probability-based merging using UMI statistic estimation reduces the sequencing-platform-specific artifacts in the plurality of families prior to determining the consensus fragment sequences, thereby producing a sequence determination of improved accuracy relative to a sequence determination performed without the probability-based merging.

2. The method of claim 1, wherein performing probability-based merging comprises iteratively:
(i) determining the relative likelihood for each of a plurality of candidate pairs of families;
(ii) selecting the candidate pair having the highest relative likelihood among all candidate pairs of families;
(iii) merging the selected pair when the highest relative likelihood exceeds the merging threshold; and
(iv) repeating (i)-(iii) until no pair exceeds the merging threshold.

3. The method of claim 2, wherein the merging threshold is dynamically adjusted based on one or more of the fragment size frequency, the UMI jumping rate, and the UMI frequency determined in the UMI statistic estimation.

4. The method of claim 1, wherein performing UMI statistic estimation comprises performing UMI statistic estimation on a subset of the plurality of families and applying results of the estimation to all families.

5. The method of claim 1, wherein determining the relative likelihood further comprises:
applying a sequencing error rate that is characteristic of the high-throughput sequencing platform, optionally wherein the sequencing error rate is 0.001; and
applying a mismatch probability, optionally wherein the mismatch probability is 0.25;
optionally wherein the sequencing error rate and/or the mismatch probability is predetermined.

6. The method of claim 1, wherein:
the merging threshold is 1; and/or
merging the first family and the second family comprises merging a smaller family of the two families into a larger family of the two families.

7. The method of claim 2, wherein performing probability-based merging comprises:
family identification and merging; and/or
duplex identification and merging;
optionally wherein performing probability-based merging comprises performing probability-based merging using a probability map.

8. The method of claim 1, wherein the plurality of families prior to probability-based merging comprises at least 10% more families than the plurality of families subsequent to probability-based merging.

9. The method of claim 1, wherein each sequence read of the plurality of sequence reads further comprises a second UMI sequence, wherein:
(i) the UMI sequence is 5′ to the fragment sequence and the second UMI sequence is 3′ to the fragment sequence; or
(ii) the UMI sequence is 3′ to the fragment sequence and the second UMI sequence is 5′ to the fragment sequence;
optionally wherein the UMI sequence and/or the second UMI sequence is 4-20 bases in length; and optionally wherein the UMI sequences are random or non-random.

10. The method of claim 1, wherein:
(1) the plurality of sequence reads comprises paired-end sequence reads and/or single-end sequence reads;
(2) the plurality of sequence reads is generated by whole genome sequencing (WGS), optionally wherein the WGS is clinical WGS (cWGS); and/or
(3) the reference sequence comprises a reference genome sequence.

11. The method of claim 1, wherein the biological sample comprises cells, cell-free DNA, cell-free fetal DNA, circulating tumor DNA, amniotic fluid, a blood sample, a biopsy sample, or a combination thereof.

12. The method of claim 1, further comprising, subsequent to probability-based merging:
aligning the consensus fragment sequence to the reference sequence;
determining a position of the consensus fragment sequence aligned to the reference sequence; and/or
identifying one or more sequence variants in the original nucleic acid molecule based on the consensus fragment sequences, wherein the one or more sequence variants are identified with improved sensitivity or specificity relative to variants identified without performing probability-based merging.

13. The method of claim 1, further comprising generating a file, a report, and/or a user interface comprising a user interface element representing, for one or more of the plurality of families, at least one of:
(i) the family;
(ii) sequence reads of the family, fragment sequences of the family, UMI sequences of the family, or combinations thereof, or
(iii) a consensus fragment sequence of the family, a position of the consensus fragment sequence aligned to the reference sequence, a consensus UMI sequence of the family, or combinations thereof.

14. The method of claim 1, wherein the fragment sequences of the plurality of sequence reads are 50 base pairs to 1,000 base pairs in length.

15. A computer-implemented method for determining a sequence of an original nucleic acid molecule, the method performed under control of one or more hardware processors and comprising:
receiving a plurality of sequence reads generated by a high-throughput sequencing platform, wherein each sequence read of the plurality of sequence reads comprises a fragment sequence and a UMI sequence;
aligning the plurality of sequence reads to a reference sequence using the fragment sequences of the sequence reads;
grouping the aligned plurality of sequence reads into a plurality of families of sequence reads based on the UMI sequences and positions of the fragment sequences aligned to the reference sequence;
performing UMI statistic estimation on the plurality of families, wherein performing UMI statistic estimation comprises determining, from the plurality of families, one or more of: fragment size frequency, UMI jumping rate, and UMI frequency;
performing probability-based merging of families of the plurality of families using results of the UMI statistic estimation, wherein performing probability-based merging comprises:
determining a relative likelihood that a first family and a second family of the plurality of families are derived from the same original nucleic acid molecule, wherein the relative likelihood is a product of:
a likelihood ratio of a unique molecule over a non-unique molecule given the fragment positions of the first family and the second family, and
a likelihood ratio of UMI transition for a unique molecule over a non-unique molecule;
determining that the relative likelihood exceeds a merging threshold; and
merging the first family and the second family into a merged family;
subsequent to performing probability-based merging, determining a consensus fragment sequence for each of one or more of the plurality of families; and
determining the sequence of the original nucleic acid molecule based on the consensus fragment sequences,
wherein the computer-implemented method constitutes a specific improvement to nucleic acid sequence determination by correcting for sequencing-platform-introduced artifacts through UMI statistic estimation and probability-based merging, producing a sequence determination of improved accuracy that is not achievable by the high-throughput sequencing platform alone.

16. The method of claim 15, wherein performing probability-based merging comprises iteratively:
(i) determining the relative likelihood for each candidate pair of families of the plurality of families;
(ii) identifying the candidate pair having the highest relative likelihood;
(iii) if the highest relative likelihood exceeds the merging threshold, merging the identified candidate pair into a merged family and updating the plurality of families; and
(iv) repeating steps (i) through (iii) until no candidate pair of families has a relative likelihood exceeding the merging threshold.

17. The method of claim 16, wherein the merging threshold is dynamically adjusted based on one or more of the fragment size frequency, the UMI jumping rate, and the UMI frequency determined in the UMI statistic estimation.

18. The method of claim 15, wherein determining the relative likelihood further comprises:
applying a sequencing error rate, optionally wherein the sequencing error rate is 0.001; and/or
applying a mismatch probability, optionally wherein the mismatch probability is 0.25;
optionally wherein the sequencing error rate and/or the mismatch probability is predetermined.

19. The method of claim 15, wherein:
the merging threshold is 1; and/or
merging the first family and the second family comprises merging a smaller family of the two families into a larger family of the two families.

20. The method of claim 16, wherein performing probability-based merging comprises:
family identification and merging; and/or
duplex identification and merging;
optionally wherein performing probability-based merging comprises performing probability-based merging using a probability map.

21. The method of claim 16, wherein the plurality of families prior to probability-based merging comprises at least 10% more families than the plurality of families subsequent to probability-based merging.

22. The method of claim 15, wherein each sequence read of the plurality of sequence reads further comprises a second UMI sequence, wherein:
(i) the UMI sequence is 5′ to the fragment sequence and the second UMI sequence is 3′ to the fragment sequence; or
(ii) the UMI sequence is 3′ to the fragment sequence and the second UMI sequence is 5′ to the fragment sequence;
optionally wherein the UMI sequence and/or the second UMI sequence is 4-20 bases in length; and optionally wherein the UMI sequences are random or non-random.

23. The method of claim 15, wherein the plurality of sequence reads comprises paired-end sequence reads and/or single-end sequence reads; and/or wherein the plurality of sequence reads is generated by whole genome sequencing (WGS), optionally clinical WGS (cWGS).

24. The method of claim 16, further comprising, subsequent to probability-based merging:
aligning the consensus fragment sequence to the reference sequence;
determining a position of the consensus fragment sequence aligned to the reference sequence; and/or
identifying one or more sequence variants in the original nucleic acid molecule based on the consensus fragment sequences, wherein the one or more sequence variants are identified with improved sensitivity or specificity relative to variants identified without performing probability-based merging.

25. The method of claim 16, further comprising generating a file, a report, and/or a user interface comprising a user interface element representing, for one or more of the plurality of families, at least one of:
(i) the family;
(ii) sequence reads of the family, fragment sequences of the family, UMI sequences of the family, or combinations thereof, or
(iii) a consensus fragment sequence of the family, a position of the consensus fragment sequence aligned to the reference sequence, a consensus UMI sequence of the family, or combinations thereof.

26. The method of claim 15, wherein the fragment sequences of the plurality of sequence reads are 50 base pairs to 1,000 base pairs in length.


SEQUENCE LISTING (5 sequences)

SEQ ID NO: 1 - DNA, 10 bp, synthetic construct
  misc_feature 1..10: note = Unique Molecular Identifier (UMI) Sequence
  source 1..10: mol_type = other DNA
catcggggag

SEQ ID NO: 2 - DNA, 10 bp, synthetic construct
  misc_feature 1..10: note = Unique Molecular Identifier (UMI) Sequence
  source 1..10: mol_type = other DNA
gagttactgg

SEQ ID NO: 3 - DNA, 10 bp, synthetic construct
  misc_feature 1..10: note = Unique Molecular Identifier (UMI) Sequence
  source 1..10: mol_type = other DNA
gcgggggaac

SEQ ID NO: 4 - DNA, 10 bp, synthetic construct
  misc_feature 1..10: note = Unique Molecular Identifier (UMI) Sequence
  source 1..10: mol_type = other DNA
gcgtgggaac

SEQ ID NO: 5 - DNA, 10 bp, synthetic construct
  misc_feature 1..10: note = Unique Molecular Identifier (UMI) Sequence
  source 1..10: mol_type = other DNA
ttaatgagct